(19) United States
(12) Patent Application Publication
(10) Pub. No.: US 2021/0401746 A1
(43) Pub. Date: Dec. 30, 2021

(54) STABLE CANNABINOID COMPOSITIONS

(71) Applicant: cbdMD, Inc., Charlotte, NC (US)

(72) Inventor: Justin D. Crumrine, Charlotte, NC (US)

(21) Appl. No.: 17/359,193

(22) Filed: Jun. 25, 2021

Related U.S. Application Data
(60) Provisional application No. 63/044,856, filed on Jun. 26, 2020.

(51) Int. Cl. A61K 9/107; A61K 31/05; B01F 3/08; A61K 36/73

(52) U.S. Cl. CPC A61K 9/107; A61K 31/05; B01F 2003/0842; A61K 36/73; B01F 3/0811

(57) ABSTRACT

The present application discloses powder and aqueous formulations. These include but are not limited to water dispersible cannabinoid formulations, especially those comprising cannabidiol (CBD), cannabigerol (CBG), and cannabinol (CBN) as well as other cannabinoids. Generally, these embodiments do not include major amounts of Tetrahydrocannabinol (THC), but certain embodiments are envisioned that do contain measurable concentrations of THC. Embodiments may include one or more emulsifiers selected from the group consisting of Tween (polysorbate) 20, Tween 60, Tween 80, Span 20, Span 60, Span 80, Poloxamer 188, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin.

### CROSS-REFERENCE TO RELATED APPLICATIONS

This application claims the benefit of U.S. Provisional Application 63/044,856 filed on Jun. 26, 2020, the disclosure of which is incorporated herein by reference.

### BACKGROUND

Cannabinoids are a class of diverse chemical compounds that act on cannabinoid receptors in cells and potentially alter neurotransmitter release in the brain. Many cannabinoids have been identified from cannabis, of which the two most prominent and intensively studied are cannabidiol (CBD) and tetrahydrocannabinol (THC). Great interest is also apparent for cannabigerol (CBG), and cannabinol (CBN). The cannabis plant, also known as hemp or marijuana, has been used throughout agricultural history as a source of an intoxicant, medicine and fiber. The medical use of cannabis is deeply rooted in history. For almost 5,000 years, cannabis and preparations produced from it have been used for applications including treating nausea, inflammation, vomiting and pain. The most studied component of the cannabis plant is a group of chemicals called cannabinoids or phytocannabinoids. Cannabinoids largely contribute to the cannabis plant's medical and recreational properties.

CBD, a cannabinoid constituent of cannabis plants, possesses anxiolytic, antipsychotic, antiemetic and anti-inflammatory properties, without exhibiting the psychoactive effects of THC. THC and CBD are synthesized in the plant as DELTA 9-tetrahydrocannabinolic acid and cannabidiolic acid from the common precursor olivetol. Both THC and CBD exert their effects by interacting with the G protein-coupled cannabinoid receptors (GPCRs), CB1 and CB2 with varying affinities. CB1 receptors are expressed in larger quantities in the brain and regions in the central nervous system, and in lower amounts in peripheral tissues; the CB2 receptors have been identified to be localized to immune cells, tonsils and the spleen. The CB1 receptors appear to play significant roles in pain perception, memory, motor regulation, appetite, mood and sleep, whereas the CB2 receptors are linked with anti-inflammation, pain reduction and reducing tissue damage. Upon activation by endocannabinoids like anandamide and 2-arachidonylglycerol (2-AG) (which are short lived), CB1 and CB2 trigger downstream cascades that help mediate homeostasis and healthy functioning. In contrast, the plant-derived cannabinoids THC and CBD that directly or indirectly interact with CB1 and CB2 with varying affinities modulate the activities of these receptors for prolonged durations.

THC is a psychoactive cannabinoid found in cannabis and mimics the action of the endogenous cannabinoid receptor ligands anandamide and 2-AG by activating both CB1 and CB2 receptors. Due to its binding to CB1 receptors which are specifically present in the central nervous system in areas associated with pain (e.g., spinal trigeminal nucleus, amygdala, basal ganglia and periaqueductal gray), THC possesses antinociceptive activity and is hence used as an analgesic agent in certain pain medications. In addition, THC has also been shown to be effective in the treatment of glaucoma, nausea, chronic pain, multiple sclerosis, epilepsy and inflammation in several pre-clinical and clinical studies.

CBD is a potential therapeutic agent for various disorders of the central nervous system including anxiety, epilepsy, schizophrenia, Parkinson's disease, Alzheimer's disease, multiple sclerosis and many more. Unlike THC, CBD does not appear to activate CB1 and CB2, and instead blocks, by complex mechanisms, the ligands that activate these receptors. Several groups have proposed that this activity not only results in the non-psychotropic effects exhibited by CBD but may also account for ameliorating some of the psychotropic effects shown by THC. In addition, by lowering the psychoactivity of THC, CBD may also potentiate some of THC's benefits by enhancing its tolerability and widening its therapeutic window. Thus, use of CBD along with THC can have distinct advantages. CBD can also inhibit or delay the reuptake and hydrolysis of endocannabinoids like anandamide and adenosine. CBD has also been hypothesized to interact with several other non-endocannabinoid signaling systems such as serotonin receptors, vanilloid receptors, GPR-55 (orphan receptors), and peroxisome proliferator activated receptors (PPARs), making it a possible “multi-target drug.” In addition to these activities, the polyphenolic ring in CBD also results in it being a potent antioxidant. All these properties have prompted the exploration of the therapeutic potential of CBD for a range of neuropsychiatric as well as inflammatory disorders; unfortunately in its most common oil form it can present formulation problems.

The oral route is the most commonly acceptable delivery mechanism for drug and nutraceauticals. However, sublingual and topical delivery is also useful, but can have limited uptake potential. Lipid solubility and molecular size are the major limiting factors for molecules to pass through biological membranes and to be absorbed systematically following oral or topical administration. High lipophilicity of cannabinoids results in poor dissolution in the aqueous environment of the gastriointestinal tract. It not only makes this class of compounds poorly absorbed systemically from oral dosage forms, but complicates the physiologiceffects. The low bioavailability of oral cannabinoids thereby restricts their therapeutic and supplement uses.

Currently, there are only a few approved oral formulations of cannabinoids commercially available for treating nausea, vomiting associated with cancer, multiple sclerosis, intractable cancer pain, etc. There is a need for novel and effective oral formulations of cannabinoids that can be applied not only to pharmaceutical, but also food, beverage and supplement applications as well as topical use.

Emulsions can be used as flavor, drug or nutraceutical delivery systems in beverages, cosmetics, or pharmaceuticals. Typical emulsions are a mixture of water and oil with the help of amphiphilic surfactants. Oil in water (O/W) emulsions can carry lipophilic compounds into suspended small oil droplets and makes the compounds “water soluble”. Given the lipophilic property of cannabinoids, emulsions are a desirable delivery systems for them. Nevertheless, the limited stability of emulsions remains a problem for practitioners. Often, with time such formulations will undergo coalescence, creaming, sedimentation, Ostwald ripening that results in phase separation into two or more liquid phases. This represents the biggest challenge in existing commercial applications. This is particularly true for compositions that may experience temperature fluctuations during storage.

Unlike emulsions, microemulsions, including micellar solutions, are often thermodynamically stable. Such microemulsions can contain other useful products, e.g., whey protein and nutraceuticals, that can confer greater stability and other health benefits. Such microemultions are sometimes transparent/translucid dispersions that can even form spontaneously without the need of high shear energy input, when the compounds are properly mixed with each other under the right conditions. The dispersed oil droplets in microemulsions can be 100 nm or less in diameter. This permits the solution to be transparent in visible light as a result of minimal visible light scattering. As a result, microemulsions can appear as clear or translucid isotropic solutions, when the formulation composition and conditions are designed properly. Since microemulsions offer the advantage of spontaneous formation, ease of manufacturing and scale-up, thermodynamic stability, and improved drug solubilization and potential for improved oral bioavailability, they are a preferred delivery systems for encapsulating cannabinoids.

A classical oil-in-water microemulsion consists of water, a co-solvent, oil and one or more surfactants and/or co-surfactants. The selection of the components, the proportion of each ingredient, and the methods of emulsification are critical for their formation. Thus, certain formulations can exhibit the unexpected quality of being more stable, more transparent, or more easily formed than a formulation that is very similar. Thus, ranges of addition and process stems can be critical in certain environments. Accordingly, the final characteristics such as optical appearance, and the organoleptic and thermodynamic time-stability of the microemulsion can be unexpectedly superior compared to other similar formulations. In addition, when such microemulsions are used as nutraceuticals or drug delivery systems in foods and beverages, they may lead to excellent shelf-life stability and a significant relative bioavailabitity increase via oral administration for the encapsulated cannabinoids.

### SUMMARY

A need exists for novel methods of preparing and delivering stabilized formulations comprising CBD and THC compositions as pharmaceutical or nutraceutical products, food and beverages, optionally containing other nutritional products. In one embodiment, the present application discloses CBD alone or with THC as pharmaceutical or nutraceutical products, topicals, food and beverages, optionally containing other nutritional, topical, or pharmaceutical products. In one aspect, the formulations are oxidatively stable, are water soluble, and provide increased bioavailability that allows for effective uptake by the body.

In one embodiment, there is provided a stable emulsion comprising a CBD oil or THC oil (or CBD and/or THC, or referred to as CBD/THC), or CBD/THC mixtures. In another embodiment, there is provided a stable, substantially clear, water soluble formulation of CBD, THC or CBD/THC mixtures.

In one embodiment, the present application discloses a stabilized, aqueous cannabis oil emulsion comprising: a) CBD and b) at least one emulsifier selected from the group consisting of Tween 20, Tween 60, Tween 80, Span 20, Span 60, Span 80, Poloxamer 188, Polysorbate 80, Polysorbate 20, Vit E-TPGS (TPGS), TPGS-1000, TPGS-750-M, Solutol HS 15, PEG-40 Hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; wherein the emulsion is stable for a period of at least 30 days when stored at about 20-30 degree ° C. In one aspect, the CBD or mixtures of CBD and THC are isolates. In another aspect, the cannabis oil is purified. As used herein, the purification of the cannabis oil may be performed using distillation such as distillation under reduced pressure, chromatography such as HPLC, crystallization, and a combination thereof. In one variation, the purified CBD and THC in the cannabis oil is greater than 20% more preferably greater that 25% in the cannabis oil, most preferably greater than 30% in cannabis oil; other embodiments envisioned include greater that 35% or greater than 40% cannabis oil.

In another aspect of the above emulsion, the emulsifier is GRAS, an approved food additive, or an otherwise legal food grade emulsifier. In one aspect, the purity of CBD is from about 20% to 98%, such as 30%, 40%, 50%, 60%, 70%, 80%, 90% or about 98%. In another aspect, the purity of the CBD is about 99%, 98%, 97%, 95%, 93%, 92%, 90%, 85%, 80% 70%, 60%, 50%, 40%, 30%, 20%, 10% or about 5%. Natural CBD oils are typically refined, purified, distilled, or extracted from plant sources, and may also contain other natural oils. The purity of the CBD noted above may be determined by HPLC methods or by gas chromatography.

In another aspect, the emulsion, such as a water emulsion, further comprises one or more co-solvents selected from the group consisting of ethanol, glycerol, propylene glycol, 1,3-propanediol, butylene glycol, erythritol, xylitol, mannitol, sorbitol, isomalt, polyethylene glycols (PEG)-400, and a combination thereof. In another aspect, the emulsion further comprises one or more vegetable oils selected from the group consisting of an arachis oil, olive oil, sesame oil, tocopherol oil, cocoa oil, coconut oil, a mineral oil, rice bran oil, or combinations thereof.

In yet another aspect, the emulsion further comprises one or more oil selected from the group consisting of Cannabis oil (hemp oil), cottonseed oil, soybean oil, sunflower oil, castor oil, corn oil, olive oil, palm oil, peanut oil, almond oil, sesame oil, rapeseed oil, peppermint oil, canola oil, palm kernel oil, hydrogenated soybean oil, medium-triglyceride, short-chain triglyceride, glyceryl esters of saturated fatty acids, glyceryl behenate, glyceryl distearate, glyceryl isostearate, glyceryl laurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and combinations thereof.

In another aspect, the emulsion further comprises one or more masking or flavoring component selected from the group consisting of natural cinnamon oil, eucalyptus oil, peppermint oil, clove oil, bay oil, thyme oil, artificial, natural or synthetic fruit flavors selected from the group consisting of vanilla, chocolate, coffee, cocoa, and citrus oil selected from the group consisting of lemon, lime, orange, grape, grapefruit, and fruit essences selected from the group consisting of apple, pear, peach, strawberry, watermelon, raspberry, cherry, plum, pineapple and apricot, or combinations thereof.

In another aspect, the emulsion further comprises one or more additives comprising: a) a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof; and b) a reducing agent selected from the group consisting of L-ascorbic acid-6-palmitate, vitamin C and ubiquinol, or mixtures thereof. In another aspect, the emulsion further comprises a metal chelator selected from the group consisting of ethylenediaminetetraacetic acid (EDTA), disodium EDTA and calcium disodium EDTA and mixtures thereof.

In another aspect of the emulsion, the range of the ratio of the emulsifier to CBD oil is between 11.0:1.0 to 1.0:1.0, 7.0:1.0 to 1.5:1.0 or 5.0:1.0 to 2.0:1.0. In one aspect, the ratio of the emulsifier to CBD oil is between about 15:1 to about 10:1, 10:1 to 5:1, 5:1 to 3:1, 3:1 to 2:1, or about 2:1 to 1:1. In one aspect of the emulsion, the ratio of the emulsifier to CBD oil is between about 15:1, 13:1, 12:1, 10:1, 9:1, 8:1, 7:1, 6:1, 5:1, 4:1, 3:1, 2:1 or about 1:1. In another aspect of the emulsion, the CBD oil concentration in the emulsion is about 10%, 9%, 8%, 7%, 5%, 3%, 2%, 1%, 0.5%, 0.1% or 0.01% or less. In one aspect, the range of the CBD oil concentration in the emulsion is from about 0.01% to 10% w/w, 1%-9% or about 2%-6%.

In another aspect, the emulsion comprises particle sizes that are less than about 500 nm, less than 300 nm, less than 200 nm, less than 100 nm, less than 80 nm, less than 60 nm; less than 40 nm; or between about 20 and 30 nm, as measured by DLS or cryo-TEM. In another variation, the emulsion comprises of particle size that is in the range of about 20 nm to 80 nm, 20 nm to 40 nm, 40 nm to 60 nm and about 60 nm to 80 nm. In another aspect, the emulsion has a measured turbidity range of about 10 to 1000, 20 to 300 or 30 to 100.

In another embodiment, there is provided a method for the preparation of any one of the above emulsions, the method comprising: a) weighing the components of the above emulsion into a reaction container; b) heating the combined emulsion to a temperature from about 25 degree ° C. to about 130 degree ° C. with agitation for a sufficient amount of time to prepare the emulsion; and c) cooling the emulsion to about 25 degree ° C. In another aspect, the preparation is performed under nitrogen or other inert atmosphere. In yet another aspect, the heating of the emulsion is performed to a temperature of about 70 degree ° C. to 100 degree ° C., or about 80 degree ° C. to 95 degree ° C. In another aspect, the cooling of the emulsion is performed using an external ice bath or equivalent. In another aspect the emulsion is prepared by the cited method, the resulting stable emulsion has a shelf stability of at least 3 months, 6 months or 12 months when stored at about 0 degree ° C. to 50 degree ° C., or about 25 degree ° C. to 35 degree ° C. In one aspect of the emulsion, resulting stable emulsion has a shelf stability of at least 3 months, 6 months or 12 months when stored at about 0-50 degree ° C., 10-40 degree ° C. or 20-30 degree ° C. In another embodiment of the above emulsions, the natural odor of the CBD emulsion is effectively masked and provides a pleasant taste for oral consumption. In another aspect of the emulsion, the oxidative stability of the emulsion is enhanced over that of a CBD mixture by at least 3 months when the CBD mixture is stored at about 0 degree ° C. to 50 degree ° C.

In another embodiment, there is provided a liquid nutritional composition selected from the group consisting of beverages, soft drinks, carbonated beverages, enhanced waters, gels, gelatins, concentrates, beverage enhancers, wherein the composition is prepared by the method comprising: a) obtaining an emulsion of the above; and b) diluting the emulsion to a desired liquid nutritional composition.

In another embodiment, the water soluble formulation further comprises a water soluble antioxidant. In another embodiment, the water soluble formulation further comprises a metal chelator. In another embodiment, the water soluble formulation further comprises a water soluble reducing agent. In another embodiment, the water soluble formulation further comprises a lipophilic antioxidant. In yet another embodiment, the water soluble formulation further comprises a lipophilic reducing agent, or a combination of each of the above.

In another embodiment, there is provided a method for stabilizing a substantially water insoluble CBD mixture in an aqueous solution comprising contacting the CBD mixture with a composition comprising a micelle-forming surfactant, a water soluble reducing agent, and a metal chelator in water, at an elevated temperature, and for a sufficient period of time to dissolve the CBD mixture. In another aspect, the micelle-forming surfactant is TPGS, TPGS-750-M or TPGS-1000. In one variation, the metal chelator is ethylenediamine tetraacetic acid. In another variation, the method further comprises contacting the aqueous solution with a metal bisulfite reducing agent.

In one embodiment, there is provided a stabilized aqueous formulation comprising a substantially water insoluble CBD mixture, a micelle-forming surfactant, a water soluble reducing agent, a metal chelator and a reducing agent, wherein the formulation remains substantially clear and stable when stored at or below room temperature for a period of at least 6 months or at least 12 months. In another embodiment, there is provided a stabilized food, beverage, pharmaceutical or nutraceutical product comprising the aqueous formulation of the above.

### Definitions

Unless specifically noted otherwise herein, the definitions of the terms used are standard definitions used in the art of organic synthesis and pharmaceutical sciences. It is intended that the embodiments, aspects and variations are to be considered illustrative and not limiting.

The term “absorption enhancer” usually refers to an agent whose function is to increase absorption by enhancing membrane permeation, rather than increasing solubility, so such agents are sometimes more specifically termed permeation enhancers. Embodiments of this invention use absorption enhancers to increase the amount of CBDs into the body.

The term “cannabinoid” is defined as one of a class of diverse compounds that act on cannabinoid receptors in cells that alter neurotransmitter release in the brain. Cannabinoid may comprise all ligands of the cannabinoid receptor and related compounds, including the endocannabinoids (produced naturally in the body by animals), the phytocannabinoids (found in cannabis and some other plants), and synthetic cannabinoids that may be manufactured artificially.

The term THC can include a number of compounds. The most potent stereoisomer occurs naturally as THC where the two chiral centers at C-6a and C-10a are in the trans configuration as the (−)-trans-isomer, and this stereoisomer is also known as dronobinol. There are seven double bond isomers in the partially saturated carboxylic ring. Tetrahydrocannabinol, such as DELTA-THC, helps reduce nausea and vomiting, which is particularly beneficial to patients undergoing chemotherapy for cancer. Patients suffering from AIDS often experience a lack of appetite, of which THC is also helpful in counteracting. THC is also useful for glaucoma relief.

CBD oils (or CBD oil) refer to cannabis extracts that have CBD as the main or primary bioactive components. Likewise, THC oils refer to cannabis extracts that have THC as the main or primary bioactive components. CBD oils can vary from about 10% to >98% of CBD, typical CBD oils used in these embodiments comprise CBD content varying from about 30% to 70%. Similarly, while THC oils can vary from 10 to >98% THC, a more typical commercial range used in this invention is from 60%-90%. Additionally, CBD oil can contain THC oil, and vice versa their relative ranges can be in an mount corresponding to the foregoing.

As used herein, the term “CBD and THC” or “CBD/THC” includes substantially pure CBD, substantially pure THC or a mixture of CBD and THC having the particular purity, CBD/THC ratio and concentrations as disclosed in the present application. For example, a composition comprising “CBD and THC” includes at least one of 1) substantially pure CBD without THC, 2) substantially pure THC without CBD, and 3) a mixture of both CBD and THC having the purity, concentrations and ratios as disclosed herein.

Emulsifiers (sometimes referred to as surfactants), are a group of surface-active agents that generally lower the surface tension of an aqueous solution and typically will promote the formation and stabilization of an emulsion. HLB value, which is an abbreviation of Hydrophile-Lipophile Balance, is an empirical expression for the relationship of the hydrophilic (“water-loving”) and hydrophobic (“water-hating”) groups of a surfactant. Emulsifier HLB values range from 1-45, while the range for nonionic emulsifiers typically is from 1-20. The more lipophilic an emulsifier, the lower its HLB value. Conversely, the more hydrophilic an emulsifier, the higher its HLB value. Lipophilic emulsifiers have greater solubility in oil and lipophilic substances, while hydrophilic emulsifiers dissolve more easily in aqueous media. In general, emulsifiers with HLB values greater than 10 or greater than about 10 are called “hydrophilic emulsifiers,” while emulsifiers having HLB values less than 10 or less than about 10 are referred to as “hydrophobic emulsifiers.” HLB values have been determined and are available for most emulsifiers. HLB values for a given emulsifier or co-emulsifier can vary, depending upon the empirical method used to determine the value. HLB values of emulsifiers and co-emulsifiers provide a rough guide for formulating compositions based on relative hydrophobicity/hydrophilicity. For example, an emulsifier typically is selected from among emulsifiers having HLB values within a particular range of the emulsifier or co-emulsifier that can be used to guide formulations. A co-emulsifier is an emulsifier acting together with another emulsifier to enhance its properties; and may be used to further reduce the surface tension of a liquid. In using such co-emulsifiers the emulsion will become further stabilized.

The term “vitamin C derivative” as used herein means any compound that releases ascorbic acid (vitamin C) in vivo or in vitro, as well as solvates, hydrates and salts thereof. The term also includes vitamin C analogs wherein one or more of the hydroxyl groups of vitamin C are substituted with another moiety and wherein the vitamin C analog essentially retains the stabilizing activity of vitamin C in vitro or in vivo. Vitamin C and its derivatives are anti-oxidants and can protect emulsions from oxidation and breakdown due to such things as UV light. Antioxidants are contemplated as additions to many of the emulsification embodiments of the inventions.

As used herein, the term “solubilizing agent” is used interchangeably with the term “surfactant” or “emulsifier”. In some embodiments, the solubilizing agent is a nonionic, amphiphilic molecule, wherein the term amphiphilic means that the molecule includes at least one hydrophobic (e.g., lipid-soluble) moiety, such as a moiety derived from a tocopherol, a sterol, or a quinone (or derived hydroquinone, such as in the case of ubiquinone and ubiquinol) and at least one hydrophilic (e.g., water soluble) moiety, such as polyethylene glycol or a simple sugar, carbohydrate or a carbohydrate derivative.

As used herein, the terms “stabilizer”, and “antioxidant”, are recognized in the art and refer to synthetic or natural substances that prevent or delay the oxidative or free radical or photo-induced deterioration of a compound, and combinations thereof. Exemplary stabilizers include tocopherols, flavonoids, catechins, superoxide dismutase, lecithin, gamma oryzanol; vitamins, such as vitamins A, C (ascorbic acid) and E (tocopherol and tocopherol homologues and isomers, especially alpha and gamma- and delta-tocopherol) and beta-carotene (or related carrotenoids); natural components such as camosol, carnosic acid and rosmanol found in rosemary and hawthorn extract, proanthocyanidins such as those found in grape seed or pine bark extract, and green tea extract. In one variation, the vitamin E includes all 8-isomers (all-rac-alpha-tocopherol), and also include d,l-tocopherol or d,l-tocopherol acetate. In one variation, the vitamin E is the d,d,d-alpha form of vitamin E (also known as natural 2R,4R′,8R′-alpha-tocopherol). In another variation, the vitamin E includes natural, synthetic and semi-synthetic compositions and combinations thereof.

The term “reducing agent” is any compound capable of reducing a compound of the present application to its reduced form. “Reducing agent” includes lipophilic (e.g., lipid-soluble) reducing agents. In one example, the lipid-soluble reducing agent incorporates a hydrophobic moiety, such as a substituted or unsubstituted carbon chain (e.g., a carbon chain consisting of at least 10 carbon atoms). “Reducing agent” also includes hydrophilic (e.g., water soluble) reducing agents. In one variation, the reducing agent that may be used in the formulation is ubiquinol. In one example, the reducing agent is a “water soluble reducing agent” when the reducing agent dissolves in water (e.g., at ambient temperature) to produce a clear solution, as opposed to a visibly cloudy, hazy or otherwise inhomogeneous mixture. In one example, the reducing agent is a “water soluble reducing agent” when it includes at least one (e.g., at least two) hydroxyl group(s) and includes a substituted or unsubstituted linear carbon chain consisting of not more 6, 8, 10, 11, 12, 13, 14 or 15 carbon atoms. An exemplary water soluble reducing agent is ascorbic acid. The term “water soluble reducing agent” also includes mixtures of vitamin C with the CBD mixture(s) of the present application. Water-soluble reducing agents can be derivatized to afford an essentially lipid-soluble reducing agent (pro-reducing agent). For example, the water soluble reducing agent is derivatized with a fatty acid to give, e.g., a fatty acid ester. An exemplary lipid-soluble reducing agent is ascorbic acid-palmitate.

The term “water soluble” when referring to a formulation or compositions of the present application, means that the formulation, when added to an aqueous medium (e.g., water, original beverage) dissolves in the aqueous medium to produce a solution that is essentially clear. In one example, the formulation dissolves in the aqueous medium without heating the resulting mixture above ambient temperature (e.g., 25 degree ° C.).

The term “aqueous formulation” refers to a formulation of the present application including at least about 5% (w/w) water. In one example, an aqueous formulation includes at least about 10%, at least about 20%, at least about 30% at least about 40% or at least about 50% (w/w) of water.

The term “pharmaceutical”, “pharmaceutical composition” or pharmaceutical formulation” encompasses “neutraceutical” (also referred to as “nutraceutical”), “neutraceutical composition” or “neutraceutical formulation”, respectively. Neutraceutical formulations or neutraceutical compositions may include a pharmaceutically acceptable carrier, such as those described herein.

The term “therapeutically effective amount” is well known in the art and, for example, refers to an amount of a formulation or composition disclosed herein that produces some desired effect at a reasonable benefit/risk ratio applicable to the medical treatment. In certain embodiments, the term refers to that amount necessary or sufficient to eliminate or reduce the medical symptoms for a period of time. The effective amount may vary depending on such factors as the disease or condition being treated, the size of the subject and the severity of the disease or condition. One of ordinary skill in the art may empirically determine the effective amount of a particular composition without undue experimentation.

The term “neutraceutical” or “nutraceutical” is a combination of the terms “nutritional” and “pharmaceutical”. It refers to a composition that is known or suspected in the art to positively affect human nutrition and/or health.

The term “beverage” describes any water-based liquid, which is suitable for human consumption (i.e., food-grade). A typical beverage of the present application is any “original beverage” in combination with the cannabinoid mixture(s) of the present application. “Original beverage” can be any beverage (e.g., any marketed beverage). The term “original beverage” includes beers, carbonated and non-carbonated waters (e.g., table waters and mineral waters), flavored waters (e.g., fruit-flavored waters), mineralized waters, sports drinks, smoothies, neutraceutical drinks, filtered or non-filtered fruit and vegetable juices (e.g., apple juice, orange juice, cranberry juice, pineapple juice, lemonades and combinations thereof) including those juices prepared from concentrates. Exemplary juices include fruit juices having 100% fruit juice (squeezed or made from concentrate), fruit drinks (e.g., 0-29% juice), nectars (e.g., 30-99% juice). The term “original beverage” also includes fruit flavored beverages, carbonated drinks, such as soft-drinks, fruit-flavored carbonates and mixers. Soft drinks include caffeinated soft drinks, such as coke (e.g., Pepsi Cola., Coca Cola.) and any “diet” versions thereof (e.g., including non-sugar sweeteners). The term “original beverage” also includes teas (e.g., green and black teas, herbal teas) including instant teas, coffee, including instant coffee, chocolate-based drinks, malt-based drinks, milk, drinkable dairy products and beer. The term “original beverage” also includes any liquid or powdered concentrates used to make beverages.

The term “cannabidiol” or “CBD” is a phytocannabinoid that is one of at least 113 active cannabinoids that have been identified in cannabis. CBD may account for up to 40% of the plant's extract, and have been considered to provide a large scope of potential medicinal applications. In another aspect, the term “Cannabinoid” also refers to a compound (such as cannabinol, THC or cannabidiol) that is found in the plant species Cannabis sativa (marijuana), and includes metabolites and synthetic analogues thereof, that may have psychoactive properties. Cannabinoids include compounds, such as THC, that have high affinity for the cannabinoid receptor, and compounds that do not have significant affinity for the cannabinoid receptor, such as cannabidiol (CBD). Cannabinoids also include compounds that have a characteristic dibenzopyran ring structure (such as in THC) and cannabinoids which do not have a pyran ring (such as cannabidiol). Cannabinoids also includes, for example, THC, CBD, dimethyl heptylpentyl cannabidiol (DMHP-CBD) and 6,12-dihydro-6-hydroxy-cannabidiol (see U.S. Pat. Nos. 5,227,537 and 4,876,276); and cannabidiol (−)(CBD) analogs. See Consroe et al., J. Clin. Pharmacol. 21:428S-436S, 1981 and Agurell et al., Pharmacol. Rev. 38:31-43, 1986, which are all incorporated herein by reference in its entirety. Accordingly, the term “cannabis oil” may comprise of these cannabinoids, including CBD, THC, 11-OH-THC and 11-NOR-9-Carboxy-THC, and other compounds as disclosed herein.

The term “clear beverage” (e.g., clear juice) means any beverage clear (e.g., transparent) to the human eye. Typical clear beverages include carbonated or non-carbonated waters, soft drinks, such as Sprite, Coke, or root beer, filtered juices and filtered beers. Typical non-clear beverages include orange juice with pulp and any of the common nut milks as well as animal milk.

The term “non-alcoholic beverage” includes beverages containing essentially no alcohol. Exemplary non-alcoholic beverages include kombuchas, those listed above for the term “beverage”. The term “non-alcoholic beverage” includes beers, including those generally referred to as “non-alcoholic beers”. In one example, the non-alcoholic beverage includes less than about 10% alcohol by volume. In other examples, the non-alcoholic beverage includes less than about 9%, less than about 8%, less than about 7%, less than about 6% or less than about 5% alcohol by volume.

The term “essentially stable to chemical degradation” refers to the CBD and/or THC and/or CBD/THC or other cannabinoid compositions, and mixtures thereof, of the present application as contained in a formulation (e.g., aqueous formulation), beverage or other composition of the present application. In one example, “essentially stable to chemical degradation” means that the composition is stable in its original form and is not converted to another species (e.g., oxidized species or any other species having an essentially different molecular structure), for example, through oxidation, cleavage, rearrangement, polymerization and the like, including those processes induced by light (e.g., radical mechanisms).

The term “essentially clear” is used herein to describe the compositions (e.g., formulations) of the present application. For example, the term “essentially clear” is used to describe an aqueous formulation or a beverage of the present application, such clarity may be assessed by the human eye. In this example, “essentially clear” means that the composition is transparent and essentially free of visible particles and/or precipitation (e.g., not visibly cloudy, hazy or otherwise non-homogeneous). In another example, clarity, haziness or cloudiness of a composition is assessed using light scattering technology, such as dynamic light scattering (DLS), which is useful to measure the sizes of particles, e.g., micelles, contained in a composition. In one example, “essentially clear” means that the median particle size as measured by DLS is less than about 100 nm, the liquid which appears clear to the human eye. In another example, “essentially clear” means that the median particle size is less than about 80 nm; less than about 60 nm; less than about 40 nm; or between about 20 and about 30 nm. For example, to prepare a sample (e.g., formulation of the present application) for a DLS measurement, the sample is typically diluted so that the concentration of the solubilizing agent in the diluted sample is low enough to give an accurate and consistent measurement . . . . In another example, the solubilizing agent (e.g., TWEEN-85, TPGS, TPGS-750-M or TPGS-1000) is present in a concentration that is above the critical micelle concentration (CMC) (i.e., the concentration that allows for spontaneous formation of micelles in water). For example, a typical CMC for TPGS in water is about 0.1 to about 0.5 mg/ml.

Alternatively, clarity, haziness or cloudiness of the composition can be determined by measuring the turbidity of the sample, which is useful when the composition is a beverage (e.g., water, soft-drink etc.). In one example, turbidity is measured in FTU (Formazin Turbidity Units) or FNU (Formazin Nephelometric Units), or is measured using a nephelometer. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the present application (e.g., a beverage of the present application) is “essentially clear” when the turbidity is not more than about 500% higher than the control (original beverage without an added cannabinoid mixture of the present application, but optionally including a solubilizing agent of the present application). In another example, a composition of the present application is “essentially clear” when the turbidity is not more than about 300% higher than the control. In yet another example, a composition of the present application is “essentially clear” when the turbidity is not more than about 200%, about 150% or about 100% higher than the control; or is “essentially clear” when the turbidity is not more than about 80%, about 60%, about 40%, about 20% or about 10% higher than the control.

The term “emulsion” as used herein refers to a cannabinoid composition and mixture of the present application emulsified (solubilized) in an aqueous medium using a solubilizing agent of the present application. In one example, the emulsion includes micelles formed between the cannabinoid composition and mixture (i.e., mixture) and the solubilizing agent. When those micelles are sufficiently small, the emulsion is essentially clear. Typically, the emulsion will appear clear to the normal human eye, when those micelles have a median particle size of less than 100 nm. In one example, the micelles in the emulsions of the present application have median particle sizes below 60 nm. In a typical example, micelles formed in an emulsion of the present application have a median particle size between about 20 and about 30 nm. In another example, the emulsion is stable, which means that separation between the aqueous phase and the cannabinoid mixture does essentially not occur (e.g., the emulsion stays clear). A typical aqueous medium, which is used in the emulsions of the present application, is water, which may optionally contain other solubilized molecules, such as salts, coloring agents, flavoring agents and the like. In one example, the aqueous medium of the emulsion does not include an alcoholic solvent, such as ethanol or methanol.

The term “flavonoid” as used herein is recognized in the art. The term “flavonoid” includes those plant pigments found in many foods that are thought to help protect the body from disease (e.g., cancer). These include, for example, epi-gallo catechin gallate (EGCG), epi-gallo catechin (EGC) and epi-catechin (EC).

The term “GRAS” as used herein is an acronym for “Generally Recognized As Safe” as defined under the Federal Food, Drug, and Cosmetic Act and subsequent regulations. GRAS is a designation indicating that a substance or chemical is generally recognized as safe among qualified experts, as having been shown to be safe for its intended use in food. A GRAS substance is excluded from the definition of a food additive.

The term “approved food additive” as used herein is as defined under the Federal Food, Drug, and Cosmetic Act and subsequent regulations. An approved food additive is an ingredient added to food for which the statutorily required food additive petition has been received, evaluated and approved by the FDA indicating that the food ingredient has been determined by FDA to be safe for its intended use in food.

The term “metal chelator” or “metal chelating moiety” as used herein refers to a compound that combines with a metal ion, such as iron, to form a chelate structure. The chelating agents form coordinate covalent bonds with a metal ion to form the chelates. The term “metal ion” as used herein refers to any physiological, environmental and/or nutritionally relevant metal ion. Such metal ions include certain metal ions such as iron, but may also include calcium, magnesium, lead, mercury and nickel. When EDTA (or disodium EDTA or calcium disodium EDTA) is used in the present application to chelate iron, the chelate forms a Fe.sup.3+ ethylene-diaminetetraacetic acid (EDTA) complex.

The term “micelle” is used herein according to its art-recognized meaning and includes all forms of micelles, including, for example, spherical micelles, cylindrical micelles, worm-like micelles and sheet-like micelles, and vesicles, formed in water, or mostly water.

The term “pharmaceutically acceptable salts” includes salts of the active compounds which are prepared with relatively nontoxic acids or bases, depending on the particular substituents found on the compounds described herein. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present application contain both basic and acidic functionalities that allow the compounds to be converted into either base or acid addition salts.

The term “THC” or its main isomer (−)-trans-DELTA 9-tetrahydrocannabinol, is the principal psychoactive constituent (or a cannabinoid) of cannabis. THC has been employed effectively for the treatment of anorexia in patients with HIV/AIDS and for refractory nausea and vomiting in patients undergoing chemotherapy. THC is an aromatic terpenoid that has very low solubility in water.

The term “tocopherol” includes all tocopherols, including alpha-, beta-, gamma- and delta tocopherol. The term “tocopherol” also includes tocotrienols. CBD can occur in up to 40% of simple cannabinoid extracts from cannabis. CBD generally occurs in the cannabis plant prior to processing as CBDA which has a carboxylic acid group. The 2-carboxylic acids of the cannabinoids, including CBD, can be decarboxylated by heat, light, or alkaline conditions to their respective decarboxylated compounds.

The phytocannabinoids have been numbered according to the monoterpenoid system or the dibenzopyran system. Many phytocannabinoids, at least 113, have been identified, most of them belonging to several subclasses or types such as the cannabigerol (CBG), cannabichromene (CBC), cannabidiol (CBD), DELTA.9-THC, DELTA.8-THC, cannabicyclol (CBL), cannabielsoin (CBE), cannabinol (CBN), cannabinodiol (CBDL) and cannabitriol (CBTL) types. As provided herein, the compositions and formulations of the present application comprising CBD or CBD/THC, as disclosed herein, may also be replaced by the cannabinoids or mixtures of cannabinoids to provide active formulations.

Function of CBD and CBDA:

CBD and CBDA have been shown effective in treating inflammation, diabetes, cancer, mood disorders (PTSD to ADD) and neurodegenerative disease such as Alzheimer's. It has been shown to have anti-convulsive, anti-anxiety, anti-psychotic, anti-nausea and anti-rheumatoid arthritic and sedative properties, and a clinical trial showed that it eliminates anxiety and other unpleasant psychological side effects. CBD does not display the psychoactive effects of THC. CBD was found in one study to be more effective than aspirin for pain relief and reducing inflammation. CBD has been shown to be a potent antioxidant as well as having neuroprotective and anti-inflammatory uses.

Formulation of cannabinoids (mainly CBD, CBG, and CBN) in an oil-in-water (OW) emulsion are of particular interest. There are many variables which mainly fall into 2 broad categories, composition and process. Many different components typically compose the finished product or even just the base formulation of most OW emulsions and OW emulsion-containing products. The end use for this group of formulations are potentially as a supplement, food additive, cosmetic, or perhaps even pharmaceutical. Liquid beverage enhancers with cannabinids and/or possibly combined with other supplements as well (ex. Melatonin, vitamins, calming herbs etc.) are also contemplated as part of this invention. The OW emulsion base formula can be spray dried into a water-dispersible powder that can be marketed as individual servings of drink mix powders. Addition of water dispersible cannabinoids to some water/aloe based cosmetics and body sprays is also contemplated as part of this invention. Different end use has a different formulation; we have many good formulations for each product and some have unexpected results. In addition, many of these formulations exhibit long term stability and are stable for more than a year. This is especially true when protected from damaging light. In a box for over a year, Tween and Span samples still remained clear and the CBD degradation was minimal, and no sedimentation or oil separation. Other solubilizers can also deliver these qualities.

Generally, OW emulsions need to be stabilized, typically achieved using surfactants, co-surfactants, weighting agents, identity of carrier oils, and modifying the relative composition of the above-mentioned components, active compounds, and water in the final formulation. Often the system also requires significant energy input to overcome the unfavorable thermodynamic surface interactions of the poorly-mixed components. This is usually added to the system through high-shear mixing (high-pressure or high-speed homogenization or ultrasonication When the system is “well-balanced” it will create nanometer (10-100 nm, diameter) sized oil droplets stably dispersed in the water. This solution will appear as a translucent clear solution, this type of emulsion can be stable indefinitely under normal conditions. If the solution is “fairly well-balanced” then the solution will appear as a turbid (e.g., skim milk) to completely opaque (e.g., whole-milk) solution and can have oil droplets in the 100-1000+ nm average diameter range. The stability of these systems can still be quite long. They can show no visual signs of instability for more than a year, or easily and completely be re-dispersed with gentle agitation. In general cooler temperatures are better, but freezing is not recommended. Preferably, the storage is at 20° C. By stability, it is generally meant that the solution does not separate, does not form precipitate, and the average particle size does not greatly increase over time due to flocculation or Oswald ripening.

A number of different stable OW emulsion systems have been developed with different end-uses in mind. These include but are not limited to:

| System | Surfactants Used | End-Use |
|---|---|---|
| Tween/Span | Tween 80 and Span 80 | Cosmetic, haircare, trans-dermal delivery, |
| | | animal sprays/creams ( I originally |
| | | designed this system for a dog hot-spot |
| | | spray.) |
| Mixed | the best are refined | Food/beverage additives, finished |
| phospholipid | sunflower lecithins from | beverages, gummies, softgels. Also could |
| | Lipoid (german company). | work in topical cosmetic and |
| | Specifically the H90 and the | pharmaceutical products. Really this one |
| | H100/H100-3 are really | can do it all. |
| | good. These are all above | |
| | 90% phospholipids and are | |
| | mostly phospaditylcholine. | |
| Sapponins | Q-naturale V200 from | Spray-dried beverage powders |
| | ingredion, Quillaja | |
| | saponaria extract | |
| phospholipid/ | Tween 80/Span 80 and | Enhanced absorption dermal formulas for |
| tween | H100 lecithin | cosmetics and pharmaceutical |
| | | applications, human and animal |

Systems utilizing surfactants trade named Tween 80 (polysorbate 80, polyoxyethylene sorbitan monoleate) and Span 80 (sorbitan monooleate) were found to create crystal clear nano-emulsions when the relative concentration of surfactants and carrier oils, and processing parameters were optimized. This system is stable for over a year at up to 30 mg/mL CBD. Such material would generally be favored for topical use, although it is “food-safe”. Such formulations would be particularly good for body moisturizing mists with CBD (or other cannabinoids), or some other topical application.

Systems based a surfactant product made from the Quillaja saponaria tree (commonly known as the Soapbark tree) are especially useful. The surfactant is turbid white but stable indefinitely when the correct ratios of surfactant, carrier oil, cannabinoids, and processing conditions are optimized. This surfactant system can hold a moderate oil load. The ratios optimized for the nanoemulsion intended for spray-drying were between 0.3 and 6 wt. % saponin surfactant. Using a 1:2.5 ratio of surfactant to hydrophobic phase was optimal. This is in stark contrast to the tween span system where an almost opposite ratio of 2.3:1 surfactant to hydrophobic phase was optimal. We were especially interested in hydrophobic wt % between 1 and 15%, which gave a final active CBD concentrations between 0.3-5% by weight. Final concentrations can be experimentally tested by spray drying and testing. The system is almost completely flavorless. Accordingly, it is especially useful as a base for the liquid water enhancer product. It is stable in the end use formulation as well with sugars, acids, flavors (in either PG or VG), and preservatives. Pasteurization embodiments are also useful embodiments.

Embodiments of the contemplated formulations can be modified/encapsulated by adding carrier molecules of maltodextrin and/or gum acacia and then spray drying into a dry powder that can be redispersed into water. Such processes require processing techniques and dryer parameter optimizations that are well within the knowledge of those of skill in this art.

Systems with Tween 80 and specific types of soy lecithin high in phosphatidylcholine (PC) lead to a clear and stable emulsion. Such formulations are great in topicals with enhanced dermal absorption rates. PC (phosphatidylcholine) is a known tight-junction modifier and formulating it with oil and cannabinoids into a nano sized droplet enhances the absorption rate.

Processing conditions and methods had a large effect on the stability of the emulsion. In general ultra-sonication is preferred to homogenization using a high-shear vertical mixer. Ultrasonic waves can deliver incredibly large localized energy on the molecular level due to the implosion of cavitation bubbles next to the tip of the ultrasonic probe (horn). There are two main draw backs to this system: (a) its difficulty in scale up, and (b) its addition of excess heat to the system. The latter is easy enough to deal with but scale up can remain a problem. Accordingly, high shear mixing is the preferred method for large scale manufacturing like using a high-throughput high pressure homogenizer. Of particular interest,
Using a UP 100S sonicator from Heilscher with either a 7 mm or 10 mm diameter probe, increasing the amplitude form 20-80% led to a decrease in required processing time to achieve the same end point droplet size. Very little difference between increasing from 80-100% amplitude was observed
In general, dispersing or dissolving all hydrophobic ingredients with themselves and the same for any hydrophilic ingredients before mixing them together is preferred for a good end emulsion.
Forming a coarse emulsion using an IKA-T50 mixer at 10k rpm, before sonication can unexpectantly lead to a decrease in the polydispersity of the resulting oil droplet diameter in the final emulsion.
Increasing the internal processing temperature of the emulsion from 30° C. to 80° C. unexpectedly led to a decrease in processing time. In one example, this change was from 7 mL per minute to 18 mL per minute, respectively.

Oil-in-Water emulsions composed of cannabis-extract, carrier oil(s), surfactant(s), and water and sometimes a co-surfactant are most commonly prepared by high-shear mixing and ultra-sonication. This is considered a base emulsion, meaning it is optimized on its own before examining the end-use stability (mixing with sugars, acids, flavors, preservatives etc.). The effects of different surfactant/surfactant systems in both identity and relative concentrations were examined. In all cases using a surfactant to hydrophobic phase ratio much outside of the optimal range led to increased turbidity and decreased stability. In general when using too little surfactant, phase separation was observed (presence of oil droplets) often accompanied by an increase in average droplet diameter. When using too much surfactant the product tastes worse, is more costly, becomes more turbid, and starts to separate and precipitate forms resulting in some visual sedimentation.

Different carrier oils were examined and various cannabinoid concentrations within the carrier oils were also looked at. The identity of the carrier oil was found to make a difference. Saturated oils have a straight chain tail, like MCT oil. Cannabinoids have the highest solubility in MCT oil (35 wt %), a straight chain saturated oil, however, perhaps due to its shorter chains (C8-C10 average) MCT's have inferior properties as carrier oils in the emulsions. Olive, sunflower or safflower oil are all high in oleic tail groups which are mono-unsaturated C18 oils. Both olive and sunflower worked similarly well but sunflower was preferred because it has light coloration and neutral taste. The max solubility of CBD in sunflower was 29-30%.

It was found for the Tween system that higher clarity was achieved using sunflower oil over MCT oil. Although not wishing to be bound by our present belief, we attribute this to surfactant matching. Both Tween 80 and Span 80 have an oleic C18 tail group, whereas MCT oil has only saturated C8/C10 tails. When using Tween 60 and Span 60, which have saturated steric C18 tail groups, there was not any observed difference between sunflower or MCT carrier oils.

The relative concentrations of all components were examined. Different processing parameters were looked at such as, order of addition, pH, temperature, relative ultrasonic energy (amplitude×horn surface area×residence time), homogenizer use and speed.

A number of different additives are especially useful in these formulations. These include Polysorbates, e.g., Tween 80, 60, 20; Polysorbitan esters—e.g., Span 80, 60, 20; Gums/Starches, for example, Gum Acacia, Modified Gum Acacia, Modified food starch (corn) as well as prehydrated versions of most of these as well; many different lecithins from soy and sunflower varying in composition and concentration of phospholipids ranging from 30-95% with preferences set forth below:

| | | Surfactant end-use levels, | |
|---|---|---|---|
| | | optimized surfactant to | |
| System | Surfactants Used | hydrophobic phase ratios | Notes |
| Tween/Span | Tween 80 and Span 80 | 4-7% surfactant, 2.3:1 | little ph dependence |
| | | | works from pH 3-9 |
| Mixed | the most preferred are refined | 1-10%, 0.6:1 | best pH 4.5-6.5 |
| phospholipid | sunflower lecithins from | | |
| | Lipoid. Specifically the H90 | | |
| | and the H100/H100-3 are | | |
| | really good. These are all | | |
| | above 90% phospholipids | | |
| | and are mostly | | |
| | phospaditylcholine. | | |
| Sapponins | Q-naturale V200 from | 1-6%, 0.4:1 | best pH 3-6.5 |
| | ingredion, Quillaja saponaria | | |
| | extract | | |
| phospholipid/ | Tween 80/Span 80 and H100 | 3-8%, 2:1 | best pH 4.5-6.5 |
| tween | lecithin | | |

Most preferably, the phospholipid composition is phosphatidylcholine. A group of molecules isolated from the Quillaja saponaria tree is also particularly useful. The main constituents of the most preferred embodiments blend are QS-18 and QS-21. These are commonly used as vaccine adjuvants and have been gaining use in the food space. The Quilliaja Saponins are the tree bark extract and are generally recognized as safe.

Co-surfactants such as Ethanol, Limonene, propylene glycol, and Glycerin are useful modifications of the embodiments of the present invention, as are carrier oils such as Medium-chain triglycerides, Olive oil (virgin), high linoleic safflower, and high linoleic sunflower oils.

We have determined that the processing conditions can provide important properties to certain embodiments. Such processing conditions include: order of addition/mixing of components, forming a coarse emulsion before sonication using a high-speed (10k+ rpm) stand homogenizer, processing time in the sonicator, and the sonicator settings (probe type, amplitude, cycling) can provide superior end products, as can the relationship of power consumption (watts) of the probe vs. time was also examined. Temperature effects on processing time and end result can also be utilized.

Example 1: Tween and Span System—only when a few different variables were optimized a crystal clear OW emulsion could be produced with concentrations of up to 3 wt % actives (CBD) and a translucent to slightly turbid solution could be produced with concentrations of up to 6 wt % actives

Example 2: Different tweens and spans were looked at, as well as how each behaved with each other. Tween 80, 60, and 20 and Span 80, 60, 20 were examined. These differ only in the lipid tail group of the surfactant. The 60 and 20 series are both saturated having a C12 or C18 tail, and the 80 series has a mono-unsaturated C18 tail group. Matching this tail group to the carrier oil and/or the fatty tail of the cannabinoids was found to make a difference in the stability of the OW emulsion.

Example 3: This system was investigated and we found that the relative concentrations of the 2 surfactants to each other was optimal at 2.3:1, Tween:Span by wt %. Or in molar ratio it is 1:1.3 Tween:Span. Concentrations much outside of this narrow range led to turbid or opaque solutions. Ratios of 0:5 through 5:0 Tween:Span wt % were investigated.

Example 4: The ratio of total surfactants to total hydrophobic content was found to be critical. Total surfactants=tween+span; total hydrophobic=carrier oil+CBD. The optimal ratio was 2.0-2.2:1 total surfactant:total hydrophobic. Lower concentrations of surfactant were found to be less stable and often phase separation was observed or oil droplets could be seen on surface of solutions. Using higher concentrations of surfactant led to cloudy or opaque solutions. Ratios of 0.25-5:1, total surfactant:total hydrophobic were examined.

Example 5: The identity of the carrier oil was found to make a difference. Saturated oils have a straight chain tail, like MCT oil. Cannabinoids have the highest solubility in MCT oil (35 wt %), a straight chain saturated oil, however, perhaps due to its shorter chains (C8-C10 average) MCT's have inferior properties as carrier oils in the emulsions. Olive, sunflower or safflower oil are all high in oleic tail groups which are mono-unsaturated C18 oils. Both olive and sunflower worked similarly well but sunflower was chosen because it has light coloration and neutral taste. The max solubility of CBD in sunflower was 29-30%.

Example 6: Once the surfactants ratio to each other and to the oil phase had been optimized. The total oil load in the solution was examined. Between 0-9% wt % led to perfectly clear solutions. And 10-19% led to slightly cloudy solutions but a laser beam could still pass through a vial of the solution, and when held up to the light it was translucent.

This system is intended for topical use in either an aqueous/aloe based spray or serum, toner, cleanser, conditioner etc. We also contemplate use as a moisturizing body mist, and have developed multiple canine formulations, one of which is a natural relief spray for a common long haired animal condition known as hot spot.

In another embodiment, the formulation comprises substantially pure CBD mixture, that is greater than 35% pure, greater than 45%, greater than 55%, greater than 65%, greater than 75%, greater than 85%, greater than 90%, greater than 95%, or greater than 98% pure.

These formulations have several advantages. First, they provide a CBD mixture in an essentially clear, aqueous solution. This formulation can enable a consumer to ingest the CBD mixture in a liquid form, for example, in a beverage, such as water. The aqueous formulations are essentially clear, which makes the formulations more appealing to a consumer.

In another aspect, the formulation may include a solubilizing agent described herein, as well as a water soluble reducing agent (also referred to as a stabilizer). The CBD and their mixture in these formulations (especially aqueous formulations) are stable with respect to chemical degradation (e.g., oxidation). In one example, the chemical stability of the CBD mixture is a result of a synergistic effect between the nature of the solubilizing agent and the water-solubility of the reducing agent (stabilizer): The solubilizing agent is an amphiphilic, nonionic surfactant, which in aqueous solutions allows the CBD mixture to be emulsified in “nanomicelles”, which typically have an average particle size of not more than 150 nm, often below 30 nm. When the CBD mixture is solubilized in the form of these small micelles, a water soluble (as opposed to lipid-soluble) reducing agent is surprisingly effective in preventing chemical degradation of the CBD mixture in an aqueous solution. For example, the addition of a water soluble reducing agent diminishes or prevents the degradation of the CBD mixture and extends its average lifetime in solution, for example by at least 5 times.

In another example, the water soluble reducing agent can be a compound with potential health benefits, such as vitamin C. The combination of two beneficial ingredients (cannabinoid mixture and stabilizer) in a single composition provides greater convenience to a consumer. Another benefit is that the surfactant supplies a nutrient in water (e.g., vitamin E, CoQ10, etc.).

The present application further provides methods of making the formulations. The formulations of the present application can be used in a variety of products, such as foods, beverages, cosmetics and skin-care products (topical application), dietary supplements (e.g., formulated in soft-gelatine capsules) and nutraceuticals. In one embodiment, the present application provides a beverage including a formulation of the present application.

The following abbreviations are used throughout the application: TPGS-polyoxyethanyl-.alpha.-tocopheryl succinate (e.g., TPGS-1000, TPGS-600). A number following one of the above abbreviations (e.g., TPGS-600) indicates an average molecular weight of the polyoxyethanyl or poly(ethylene glycol) (PEG) moiety of the compound. A number followed by the abbreviation “Me” or “M” (e.g., TPGS-750-M or TPGS-1000Me) indicates a polyoxyethanyl moiety capped with a methyl group (methoxypolyoxyethanyl or mPEG). Formulations:
An alternative embodiment includes the above ingredients, but may rely on more than one solubilizing agent within any given formulation; i.e., a combination of surfactants (e.g., TPGS, TPGS-1000 or TWEEN-85, in any ratio). In one aspect of the formulation, the solubilizing agent is selected from the group consisting of TPGS (polyoxyethanyl-.alpha.-tocopheryl succinate), TPGS-1000 (D-alpha-tocopheryl polyethylene glycol 1000 succinate) and combinations thereof.

In yet another aspect, the solubilizing agent is selected from the group consisting of Poloxamer 188, Polysorbate 80, Polysorbate 20, Vit E-TPGS (TPGS), TPGS-750-M, TPGS-1000, Solutol HS 15, PEG-40 Hydrogenated castor oil (Cremophor RH40), PEG-35 Castor oil (Cremophor EL), PEG-8-glyceryl capylate/caprate (Labrasol), PEG-32-gut may rely nceryl laurate (Gelucire 44/14), PEG-32-glyceryl palmitostearate (Gelucire 50/13); Polysorbate 85, Polyglyceryl-6-dioleate (Caprol MPGO), mixtures of high and low HLB emulsifiers; Sorbitan monooleate (Span 80), Capmul MCM, Maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), Propylene glycol monolaurate (e.g., Capmul PG-12 or Lauroglycol 90), polyglyceryl-3 dioleate (Plurol Oleique CC497), polyglyceryl-3 diisostearate (Plurol Diisostearique) and Lecithin with and without bile salts, or combinations thereof. In another aspect, the water soluble or water-insoluble reducing agent is selected from the group consisting of L-ascorbic acid-6-palmitate, vitamin C and its salts, alpha, beta, gamma and delta tocopherol or mixtures of tocopherol and alpha, beta, gamma, and delta-tocotrienols or mixtures thereof.

In one aspect of the above formulation, the metal chelator is selected from the group consisting of ethylenediaminetetraacetic acid (EDTA), disodium EDTA and calcium disodium EDTA and mixtures thereof. In another aspect, the bisulfite is sodium bisulfite, potassium bisulfite, sodium metabisulfite or potassium metabisulfite. In another aspect of the formulation, when dissolved in water, a metal chelator provides a solution with a clarity range of about 1,000 to 20 NTU, 100 to 20 NTU, 55 to 35 NTU or about 20 to 35 NTU. In another aspect of the formulation, when dissolved in water, a metal chelator provides a solution that remains stable toward degradation when stored at or below room temperature for a period of at least 6 months or at least 12 months.

In another aspect of the above, the emulsion, when dissolved in water, provides a solution with a clarity range of about 1,000 to 20 NTU, 100 to 20 NTU or about 20 to 35 NTU, and wherein the solution remains stable toward degradation when stored at or below room temperature for a period of at least 6 months or at least 12 months.

In one aspect of the above, there is provided a stabilized beverage, pharmaceutical or nutraceutical product comprising the stabilized powder composition of the above. In one aspect, the stabilized powder composition of the present application, wherein the solution, suited for human consumption is further treated for the inactivation of microbes by a process selected from the group consisting of pasteurization, aseptic packaging, membrane permeation or combinations thereof.

PEG is usually a mixture of oligomers characterized by an average molecular weight. In one example, the PEG has an average molecular weight from about 200 to 5000, from 500 to 1500, from 500 to 800 or about 900 to 1200. In one example, the PEG is PEG-600 or is PEG-750. Both linear and branched PEG moieties can be used as the hydrophilic moiety of the solubilizing agent in the practice of the invention. In one aspect, PEG has between 1000 and 5000 subunits, 1000 subunits, between 100 and 500 subunits, between 10 and 50 subunits, between 1 and 25 subunits, between 15 and 25 subunits, between 5 and 100 subunits or between 1 and 500 subunits.

In one aspect, the ratio of the cannabinoid mixture to the solubilizing agent is from about 1:0.1 (w/w), 1:0.3, or a range of 1:0.3 (w/w) to 1:20 (w/w); or from 1:1 (w/w) to 1:20 (w/w), from 1:1 (w/w) to 1:10 (w/w); from 1:1.3 (w/w) to 1:5 (w/w), from 1:2 (w/w) to 1:4 (w/w), or is about 1:3 (w/w). In another variation, the ratio of the CBD mixture to the solubilizing agent is from about 1:0.1 (w/w) to 1:0.3 (w/w), 1:0.3 (w/w) to 1:1 (w/w), or from 1:0.5 (w/w) to 1:2 (w/w).

In one embodiment, a water soluble reducing agent contained in the formulation (e.g., aqueous formulation) protects the cannabinoid mixture from chemical degradation (e.g., oxidative and/or light-induced processes). For example, addition of vitamin C, a water soluble vitamin C derivative, or a water-insoluble version of vitamin C to a formulation containing cannabinoid(s) and TPGS serve to prolong the chemical stability of the cannabinoid mixture in the aqueous formulation for at least several weeks. In other embodiments, the water soluble reducing agent (e.g. based on vitamin C) is added to the formulation in an amount sufficient to both reduce and stabilize the cannabinoid mixture after reduction.

In one example, according to any of the above embodiments, the formulation is an aqueous formulation and includes at least about 5% (w/w) of water, at least 10%, at least 20%, at least 30%, at least 40% or at least 50% (w/w) of water. In another example, the aqueous formulation includes more than 50% (w/w) of water. For example, the aqueous formulation includes at least about 55%, at least 60%, at least 65%, at least 70%, at least 75% or at least 80% (w/w) of water. The aqueous formulation may include more than 80% (w/w) water. For example, the aqueous formulation includes at least about 85%, at least 90%, at least 92%, at least 94% or at least 96% (w/w) of water.

In one embodiment, the aqueous formulation is essentially clear (e.g., free of visible precipitation, cloudiness or haziness). In another example, the cannabinoid mixture(s) are formulated with TPGS resulting in an aqueous formulation that, likewise, is essentially clear. Clear formulations can be colored. In one example, the formulation is essentially clear when the micelles have a particle size below the visible size (e.g., below 150 nm). The micelles formed by the solubilizing agent containing the cannabinoid mixture(s) have a median (average) particle size of less than about 100 nm. In another example, the micelles formed between the cannabinoid mixture(s) and the solubilizing agent, have a median particle size of less than about 90 nm, less than about 80 nm, less than about 70 nm or less than about 60 nm. In a further example, the micelles formed between the cannabinoid mixture(s) and the solubilizing agent, have a median particle size of less than about 50 nm, less than about 40 nm or less than about 30 nm. In another exemplary embodiment, the average particle size is from about 7 nm to about 90 nm, from about 5 nm to about 70 nm, from about 10 nm to about 50 nm, from about 10 nm to about 30 nm, or from about 7 nm to about 10 nm. In a particular example, the micelles formed between the CBD mixture and the solubilizing agent, have a median particle size between about 30 nm and about 10 nm (e.g., about 25 nm).

A co-solvent is a substance that is added to a mixture of two or more separate substances that are typically immiscible, to make them mixable. Co-solvents are added to increase the solvent power of the primary substance in the mixture. In emulsion systems, co-solvent also mean the substances that are used to attribute to an increase in the solvent capacity of the formulation for incorporated drugs or nutraceuticals and help the dispersion of a system that contains a high proportion of water soluble surfactants.

The popular food grade co-solvents used include ethanol, glycerol, propylene glycol, 1,3-propanediol, butylene glycol, erythritol, xylitol, mannitol, sorbitol, isomalt, polyethylene glycols (PEG)-400, and a combination thereof.

Vegetable oils are used to aid the solubility of cannabinoids into the oil phase of emulsions. The oily fraction used can be selected from the group consisting of Cannabis oil (hemp oil), coconut oil, cottonseed oil, soybean oil, sunflower oil, castor oil, corn oil, olive oil, palm oil, peanut oil, almond oil, sesame oil, rapeseed oil, peppermint oil, canola oil, palm kernel oil, hydrogenated soybean oil, medium-triglyceride, short-chain triglyceride, glyceryl esters of saturated fatty acids, glyceryl behenate, glyceryl distearate, glyceryl isostearate, glyceryl laurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and the combination thereof.

Flavor oils suitable for preparing the microemulsion of this invention basically used to mask the flavor of cannabinoids and the unpleasant tastes of emulsifiers. They refer to a variety oils that contains one or more volatile compounds. Flavors may be chosen from synthetic flavors, flavoring oils and oil extracts derived from plants, leaves, flowers, fruits and combinations thereof. In this invention, flavor oils are selected from a list of natural cinnamon oil, peppermint oil, clove oil, bay oil, thyme oil, also from artificial, natural or synthetic fruit flavors such as vanilla, chocolate, coffee, cocoa and citrus oil, including lemon, lime, orange, grape, grapefruit, and fruit essences including apple, pear, peach, strawberry, watermelon, raspberry, cherry, plum, pineapple and apricot.

Stabilizers are additives used to help maintain emulsions or prevent degeneration in beverages. Among the most common stabilizers are Acai gum (Gum Arabic), Agar-agar, ammonium alginate, calcium alginate, Carob bean gum (Locust bean gum), Chondrus extract (Carrageenan), ghatti gum, guar gum, pectin, potassium alginate, sodium alginate, sterculia gum (karaya gum), tragacanth, gelatin, lecithin, mono-glycerides, di-glycerides, maltodextrin, xanthan gum, proplylene glycol alginates (PGA), microcrystalline cellulose, sodium carboxymethyl cellulose, Purity Gum 2000 (modified starch), pectin, carrageenan, casein and inulin.

Antioxidants can prevent or slow down the oxidation of bioactive compounds and lipids that are used in beverages. Certain antioxidants, natural or synthetic, include Vitamin A, Vitamin C, Vitamin E (tocopherols), coenzyme Q10, alpha-carotene, astaxanthin, canthaxanthin, cyaniding, quercetin, lutein, lycopene, zeaxanthin, butylated hydroxyanisole (BHA), butylated hydroxytoluene (BHT), propyl gallate, sodium ascorbate, calcium ascorbate, fatty acid esters of ascorbic acid, octyl-gallate, dodecyl gallate, erythrorbic acid, sodium erythorbate, dodecyl gallate, tertiary-butyl hydrochinone (TBHQ), citric acid, 4-hexylresorcinol.

Chelating agents bind metal ions so that contribute to the stabilization of food color, aroma and texture. Common chelating agents are citric acids, EDTA with its Na- and Ca-salts, oxystearin, orthophosphoric acid, sorbitol, tartaric acid including its Na- and K-salts and thiosulfuric acid with its Na-salt.

In another example, the aqueous formulation does not include an alcoholic solvent, although such inclusion is possible when part of the solubilizing agent (e.g., as in Cremophore, which contains ethanol). Alcoholic solvents may include solvents, such as ethanol, methanol, propanol, butanol and higher alcohols. Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of cannabinoid mixture(s) is at least about 20 mg/mL and can be as high as about 60, 80, 100 or more than about 100 mg/mL. In one example, the concentration of cannabinoid mixture(s) in the aqueous formulation of the present application is at least about 1 mg/mL, at least 5 mg/mL, at least 10 mg/mL, at least 20 mg/mL, at least 30 mg/mL, at least 40 mg/mL, at least 50 mg/mL, at least 60 mg/mL, at least 70 mg/mL or at least 80 mg/mL, at least 85 mg/mL, at least 90 mg/mL, at least 95 mg/mL or at least 100 mg/mL, at least 110 mg/mL, at least 120 mg/mL, at least 130 mg/mL, at least 140 mg/mL, at least 50 mg/mL, at least 160 mg/mL, at least 170 mg/mL, at least 180 mg/mL, at least 190 mg/mL or at least about 200 mg/mL. In another example, the concentration of cannabinoid mixture(s) in the aqueous formulation is greater than 200 mg/mL.

In one embodiment, the present application provides a water soluble formulation comprising bioactive agent or mixtures of bioactive agents as disclosed herein, a water soluble reducing and/or anti-oxidizing agent, water-insoluble reducing and/or anti-oxidizing agent, a solubilizing agent, a metal chelating agent, and a bisulfite salt or a metabisulfite salt.

In particular variations of each of the above aspects and embodiments, the formulation may comprise the cannabinoid mixture(s) and TPGS-1000; natural, non-natural and synthetic surfactants and mixtures of surfactants, including, for example, two or more surfactants of differing structural types (e.g., TPGS-1000 and Tween-80), two or more surfactants from within the same structural class (e.g., TPGS-1000+TPGS-600). In another variation, the formulations may also comprise any of the above combinations as their free alcohols, or as their ether or ester derivatives (of their PEG portion). In another particular variation, the formulations may also comprise antioxidants that are lipophilic in nature (e.g., vitamin C palmitate), hydrophilic in nature (e.g., vitamin C), and any combinations of these, including more than one of each in any formulations. In another particular variation, the formulations may also comprise chelating agents that are lipophilic in nature, hydrophilic in nature (e.g., EDTA, HEDTA, DTPA and NTA), and any combinations of these, and in any number (i.e., more than one of each in any formulation) or ratio. In another particular variation, the formulations may also comprise salts such as salts that are lipophilic in nature (e.g., ammonium salts), hydrophilic in nature, and any combinations of these, and in any number (i.e., more than one of each in any formulation) or ratio.

In one example according to any of the above embodiments, the cannabinoid mixture(s) formulation is essentially stable to chemical degradation. In one example, the cannabinoid mixture(s) is essentially stable for at least 30, 60, 180 days, or at least 6 months, 9 months or 12 months, when stored at a temperature below about 25 degree ° C. (e.g., 4 degree ° C. or 10 degree ° C.). Typically, the formulations are stored at about 4 degree ° C. At this temperature, the formulations are stable for at least 90 days, at least 6 months or at least 12 months.

Another advantage of the above formulations is that they can be light in color or substantially colorless. Light and transparent formulations have greater consumer appeal and can be colored by additional components added to the mixture. In another example, the cannabinoid mixture(s) are emulsified in the formulation in the form of micelles that include the cannabinoid mixture(s) and the solubilizing agent. In a typical emulsion of the present application, the micelles are small in size, and are between about 10 and 30 nm. In another example, the small size of the micelles causes the emulsion to be essentially clear in appearance even at high compound concentrations (e.g., 40, 60, 80 or 100 mg/mL). In one example, the cannabinoid mixture(s) concentration in the aqueous formulations is at least about 20 mg/mL and can be as high as 60, 80, 100 or more than 100 mg/mL.

In another example, the present application provides a mixture between a formulation of the present application (e.g., a water soluble formulation) and an original beverage to create a beverage. Exemplary original beverages are described herein and include carbonated or non-carbonated waters, flavored waters, soft drinks and the like. In one example, the mixture (beverage of the present application) includes between about 1 mg/L and about 1000 mg/L of solubilized cannabinoid mixture(s). In another example, the mixture includes between about 10 mg/L and 500 mg/L of solubilized cannabinoid mixture(s), between 10 mg/L and 450 mg/mL, between 10 mg/L and 400 mg/mL, between 10 mg/L and 350 mg/mL, between 10 mg/L and 300 mg/mL, or between 10 mg/L and 250 mg/mL of solubilized cannabinoid mixture(s). In a further example, the mixture includes between about 20 mg/L and about 250 mg/L, between 20 mg/L and 200 mg/mL, between 20 mg/L and 150 mg/mL, between 20 mg/L and 100 mg/mL, or between 20 mg/L and 80 mg/mL, between 20 mg/L and 60 mg/mL, or between 20 mg/L and 40 mg/mL of solubilized cannabinoid mixture(s). In one aspect, the beverage may comprise of about 1,000 mg or less of solubilized cannabinoid mixture(s), 500 mg or less, or 250 mg or less of solubilized cannabinoid mixture(s). In one aspect, the beverage may comprise of a range of about 10 mg to about 500 mg per serving. In another aspect, the beverage may comprise of a range of about 25 mg to about 500 mg per serving. In a further example, the beverage further includes a coloring agent and/or a flavoring agent. It is possible to add one or more fruit and/or vegetable juice concentrates and/or flavor improvers to the beverage. Whey proteins as well as nutraceuticals such as creatine and beta alanine can be incorporated into such beverages and they can be sweetened through the use of sugar alcohols such as dextrin.

In yet another example according to any of the above embodiments, the cannabinoid mixture(s) can be solubilized and stabilized in the beverage. For example, the beverage is essentially free of cannabinoid mixture(s) precipitation. The beverage may be essentially clear. Clarity of a beverage can be assessed using turbidity measurements. In one example, the turbidity of the cannabinoid mixture(s) beverage is comparable (e.g., not more than 5 times) of the turbidity of the control beverage. In one example, the turbidity of the beverage is not more than about 500%, not more than 400%, not more than 300% or not more than about 200% higher than the turbidity of the control, not more than about 180%, not more than about 160%, not more than about 140%, not more than about 120% or not more than 100% higher than the turbidity of the control. The turbidity is 100% higher than the control, when the turbidity of the beverage is twice as high as the turbidity of the control.

In another example, the turbidity of the cannabinoid mixture(s) beverage is stable over time. For example, the turbidity of the beverage is stable over a period of at least 60 days, at least 90 days, or at least 180 days when the beverage is stored at ambient temperature (e.g., below about 25 degree ° C.). In addition, the beverage can be enriched with vitamins. In one example, the beverage includes at least one B vitamin. Exemplary B-vitamins include vitamin B1, vitamin B2, vitamin B3, vitamin B5, vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the cannabinoid mixture(s).

Methods of making the above solubilizing agents are known in the art as disclosed in U.S. Pat. Nos. 6,045,826, 6,191,172, 6,632,443 and WO 96/17626, all incorporated by reference in their entirety. The soft gel capsules of the present application (based on a soft gel capsule weight of from about 900 mg to about 1200 mg) include a solubilizing agent from about 1% to about 30% by weight, from 5% to 30% (w/w), from 8% to 20% of a solubilizing agent, such as TPGS, TPGS-750-M or TPGS-1000.

In another embodiment, the water soluble reducing agent is vitamin C, a water soluble vitamin C derivative (e.g., a salt), or a combination thereof. In one embodiment, the compositions are selected from ascorbic acid (vitamin C), a vitamin C derivatives, salts thereof and combinations thereof. In one embodiment, the vitamin C salt, or salt of a vitamin C derivative is an edible (e.g., pharmaceutically acceptable) salt, such as a calcium, sodium, magnesium, potassium and zinc salt. Mixed salts of vitamin C or a vitamin C derivative are also within the scope of the present application. The compositions may include one or more vitamin C derivative. The vitamin C derivative can be any analog of vitamin C. Exemplary vitamin C derivative include those in which at least one of the hydroxyl groups of the ascorbic acid molecule (e.g., 2-OH, 3-OH, 5-OH, 6-OH) is derivatized with a modifying group (see e.g., U.S. Pat. No. 5,078,989, which is incorporated in its entirety into this application). In another embodiment, the compositions may include vitamin C as well as at least one vitamin C derivative.

In another embodiment, the stabilizer is in excess in relation to the CBD mixture, or the CBD mixture is in excess of the stabilizer. In another exemplary embodiment, the ratio of the CBD mixture to the stabilizer is from about 1:1 (w/w) to about 1:6 (w/w), from 1:1 (w/w) to 1:5 (w/w), from 1:1.3 (w/w) to 1:3 (w/w), from 1:2 (w/w) to 1:4 (w/w), or about 1:3 (w/w). In another embodiment, the ratio of the stabilizer to the CBD mixture is from about 1:1 (w/w) to about 1:6 (w/w), from 1:1 (w/w) to 1:5 (w/w), from 1:1.3 (w/w) to 1:3 (w/w), from 1:2 (w/w) to 1:4 (w/w) or about 1:3 (w/w).

In another embodiment, the stabilizer is vitamin C or a vitamin C derivative. In one example, the vitamin C or the vitamin C derivative is used in a molar excess in relation to the CBD mixture. In another exemplary embodiment, the ratio of the CBD mixture to vitamin C or vitamin C derivative is from about 1:1 (w/w) to 1:6 (w/w), from 1:1 (w/w) to 1:10 (w/w), from 1:1.3 (w/w) to 1:5 (w/w), from 1:2 (w/w) to 1:4 (w/w), or about 1:3 (w/w).

In another embodiment, the metal chelator, chelating agent or metal chelating moiety is a chelator that has demonstrated affinity metal ions. Such metal ions include iron, but may also include calcium, magnesium, lead, mercury and nickel. In one aspect, the chelator is EDTA or ethylenediaminetetraacetic acid disodium salt dihydrate and the metal ion is iron (II) or iron (III). In one aspect, the metal ion is iron (III). In one embodiment, the formulations of the present application include from about 0.001% to about 0.01% by weight of the chelator relative to the CBD mixture (w/w), from 0.01% to 0.1%, from 0.1% to 0.5%, from 0.5% to 1.0%, from 1.0% to 2.0%, from 2.0% to 4.0%, from 4.0% to 6.0%, or about 4% of the chelator relative to the CBD mixture. In another embodiment, the formulations of the present application include from about 6.0% to about 10.0% by weight of the chelator relative to the cannabinoid mixture(s) (w/w), from 10.0% to about 15%, or from 15% to about 20% by weight of the chelator relative to the cannabinoid mixture(s).

The formulations described herein (either aqueous or non-aqueous) can further include ingredients useful to stabilize the composition, promote the bioavailability of the cannabinoid mixture(s). Additives of the present formulations may include one or more alternative solubilizing agents, pharmaceutical drug molecules, antibiotics, sterols, vitamins, provitamins, carotenoids (e.g., alpha and beta-carotenes, cryptoxanthin, lutein and zeaxanthin), phospholipids, L-carnitine, starches, sugars, fats, stabilizers, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

In one embodiment, the formulation may further comprise gelatin, sorbitol, glycerin or any ester derivatives therefrom. In another embodiment, the formulation further comprises polysorbate 80, hydroxylated lecithin, medium chain triglycerides, annato seed extract, rice bran oil, carotenoids, titanium dioxide, suspending agents such as silica (silicon dioxide), riboflavin or mixtures thereof. Other additives can be incorporated into the present formulations including phospholipids, L-carnitine, starches, sugars, fats, stabilizers, amino acids, proteins, flavorings, coloring agents, hydrolyzed starch(es) or combinations thereof.

Vitamin(s) in a unit dosage form of the present application are present in amounts ranging from about 5 mg to about 500 mg, 10 mg to 400 mg or from about 250 mg to 400 mg. Most specifically, the vitamin(s) is present in an amount ranging from about 10 mg to 50 mg. For example, B vitamins are usually incorporated in the range of about 1 milligram to 10 milligrams, i.e., from about 3 micrograms to 50 micrograms of B12. Folic acid, for example, is generally incorporated in a range of about 50 to 400 micrograms, biotin is generally incorporated in a range of about 25 to 700 micrograms and cyanocobalamin is incorporated in a range of about 3 micrograms to 50 micrograms.

Mineral(s) in a unit dosage form of the present application are present in an amount ranging from about 25 mg to about 1000 mg, from about 25 mg to about 500 mg, or from about 100 mg to about 600 mg. In the formulations, the additional components are usually a minor component (from 0.001% to 20% by weight or preferably from 0.01% to 10% by weight) with the remainder being various vehicles or carriers and processing aids helpful for forming the desired dosing form.

The present application provides pharmaceutical formulations comprising a formulation of the present application and a pharmaceutically acceptable carrier. Pharmaceutical formulations include nutraceutical formulations. An exemplary unit dosage form (e.g., contained in a soft gel capsule) includes a pharmaceutical grade cannabinoid mixture(s) in an amount of about 1% to about 30% by weight; from about 3% to about 20% (w/w), or from about 5% to about 20% of a cannabinoid mixture(s). Typically, soft-gel formulations include from about 5% to about 30% (w/w) of cannabinoid mixture, from about 15% to about 40% (w/w) solubilizing agent (e.g., TPGS or TPGS-1000), from 30% to 60% (w/w) lipophilic carrier (e.g., fish oil) and from 1% to 10% (w/w) viscosity enhancer (e.g., beeswax). In another embodiment, the soft gel capsule of the present application includes cannabinoid mixture(s), vitamin C, solubilizing agent (e.g., TPGS or TPGS-1000), beeswax and a lipophilic carrier (e.g., fish oil). In another embodiment, the cannabinoid mixture(s) are combined with a solubilizing agent useful to improve the bioavailability of the cannabinoid mixture(s). Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof.

The pharmaceutical composition can be prepared according to known methods. Formulations are described in detail in a number of sources, which are well known and readily available, such as the US Pharmacopeia. For example, Remington's Pharmaceutical Science by E. W. Martin describes such formulations. The compositions of the subject present application are formulated such that an effective amount of the cannabinoid mixture(s) is provided in the composition. Pharmaceutical compositions are provided which comprise, an active ingredient as described above, and an effective amount of one or more pharmaceutically acceptable excipients, vehicles, carriers or diluents. Examples of such carriers include ethanol, dimethyl sulfoxide, glycerol, silica, alumina, starch, and equivalent carriers and diluents. Acceptable carriers can be either solid or liquid. Solid form preparations include powders, tablets, pills, capsules, cachets, suppositories and dispersible granules. A solid carrier can be one or more substances, which may act as diluents, flavoring agents, solubilizing agents, lubricants, suspending agents, binders, preservatives, tablet disintegrating agents or encapsulating materials.

For oral administration, the pharmaceutical compositions can take the form of tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in conventional manner.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. The unit dosage can be a liquid based preparation or formulated to be incorporated into solid food products, chewing gum, or lozenges. Pharmaceutically acceptable salts (counter ions) can be prepared by ion-exchange chromatography or other methods as are well known in the art. The formulations of the present application may be adapted to the route of administration. Those skilled in the art will recognize various synthetic methodologies that may be employed to prepare non-toxic pharmaceutical formulations incorporating the compounds described herein. A wide variety of non-toxic pharmaceutically acceptable solvents that may be used to prepare solvates of the compounds of the present application, such as water, ethanol, propylene glycol, mineral oil, vegetable oil and dimethylsulfoxide (DMSO).

The compositions of the present application may be administered orally, topically, parenterally, sublingually, or rectally in dosage unit formulations containing conventional pharmaceutically acceptable carriers, adjuvants and vehicles. The best method of administration may be a combination of methods. The term parenteral as used includes subcutaneous injections, intradermal, intravascular (e.g., intravenous), intramuscular, spinal, intrathecal injection or like injection or infusion techniques. The formulations are in a form suitable for oral use, such as tablets, troches, lozenges, aqueous or oily suspensions, dispersible powders or granules, emulsion, hard or soft capsules, soft gel capsules, or syrups or elixirs. The formulations may be prepared according to any method known in the art for the manufacture of pharmaceutical formulations and nutraceuticals, and such compositions may contain one or more agents selected from the group consisting of sweetening agents, flavoring agents, coloring agents and preserving agents to provide pharmaceutically palatable preparations. Tablets may contain the active ingredient in admixture with non-toxic pharmaceutically acceptable excipients that are suitable for the manufacture of tablets. These excipients may be inert diluents, such as calcium carbonate, sodium carbonate, lactose, calcium phosphate or sodium phosphate; granulating and disintegrating agents, such as corn starch, or alginic acid; binding agents, such as starch, gelatin or acacia; and lubricating agents, such as magnesium stearate, stearic acid or talc. The tablets may be uncoated or they may be coated by known techniques to delay disintegration and absorption in the gastrointestinal tract and thereby provide a sustained action over a longer period. See, for example, the processes describing microencapsulation for controlled release found in U.S. Pat. Nos. 5,846,566 and 6,022,562, which are incorporated by reference in their entirety. Other time delay material such as glyceryl monostearate or glyceryl distearate may be employed. Formulations for oral use may also be hard gelatin capsules wherein the active ingredient is mixed with an inert solid diluent, such as calcium carbonate, calcium phosphate or kaolin, or as soft gelatin capsules wherein the active ingredient is mixed with water or an oil medium, such as peanut oil, liquid paraffin or olive oil. Aqueous suspensions contain the active materials in admixture with excipients for the manufacture of aqueous suspensions. Such excipients are suspending agents, such as sodium carboxymethylcellulose, methylcellulose, hydroxypropylmethyl cellulose, sodium alginate, polyvinylpyrrolidone, gum tragacanth and gum acacia; and dispersing or wetting agents, which may be a naturally-occurring phosphatide, such as lecithin, or condensation products of an alkylene oxide with fatty acids, such as polyoxyethylene stearate, or condensation products of ethylene oxide with long chain aliphatic alcohols, such as heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, such as beeswax, hard paraffin or cetyl alcohol. Sweetening agents and flavoring agents may be added to provide palatable oral preparations. These compositions may be preserved by the addition of an anti-oxidant such as ascorbic acid. Dispersible powders and granules suitable for preparation of an aqueous suspension by the addition of water provide the active ingredient in admixture with a dispersing or wetting agent, suspending agent and one or more preservatives. Additional excipients, for example sweetening, flavoring and coloring agents, may also be present.

In one embodiment, the formulations may also be in the form of oil-in-water emulsions and water-in-oil emulsions. The oily phase may be a vegetable oil, such as those described herein. Of particular interest are olive oil or arachis oil, or a mineral oil, such as liquid paraffin or mixtures of these. Suitable emulsifying agents may be naturally-occurring gums, such as gum acacia or gum tragacanth; naturally-occurring phosphatides, such as soy bean, lecithin, and esters or partial esters derived from fatty acids and hexitol; anhydrides, such as sorbitan monooleate; and condensation products of the partial esters with ethylene oxide, such as polyoxyethylene sorbitan monooleate. The emulsions may also contain sweetening and flavoring agents. Syrups and elixirs may be formulated with sweetening agents, for example glycerol, propylene glycol, sorbitol or sucrose. The formulations may also contain a demulcent, a preservative and flavoring and coloring agents. The formulations may be in the form of a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, for example as a solution in 1,3-butanediol. The acceptable vehicles and solvents include water, Ringer's solution and isotonic sodium chloride solution. Sterile, fixed oils may be employed as a solvent or suspending medium. Any bland fixed oil may be employed including synthetic mono- or diglycerides; or fatty acids such as oleic acid for preparation of injectables.

For administration to non-human animals, the formulations of the present application may be added to the animal's feed or drinking water. It may be formulated for animal feed and drinking water products so that the animal takes in an appropriate quantity of the compound in its diet or added as a spray to control such conditions as hot spot. The compound may be a composition as a premix for addition to the feed or drinking water. The composition can also be added as a food or drink supplement for humans. Dosage levels (with respect to cannabinoid mixture(s) or composition) of the order of from about 1 mg to about 250 mg per kilogram of body weight per day are useful. For example, a dosage level from about 25 mg to about 150 mg per kilogram of body weight per day, are useful. The amount of active ingredient that may be combined with the carrier materials to produce a single dosage form will vary depending upon the condition being treated and the particular mode of administration. Dosage unit forms will generally contain between from about 1 mg to about 500 mg of the cannabinoid mixture(s) and carotenoids (e.g., astaxanthin, fucoxanthin, cantaxanthin and the like). For example, dosage unit forms of about 1 mg to 250 mg, 1 mg to 100 mg or 1 mg to about 80, 60, 40, 20 or 10 mg are useful. Frequency of dosage may also vary depending on the compound used and the particular disease treated. For treatment of most disorders, a dosage regimen of 4 times daily or less may be used. The specific dose level for any particular patient will depend upon a variety of factors including the activity of the specific compound employed, the age, body weight, general health, sex, diet, time of administration, route of administration and rate of excretion, drug combination and the severity of the particular disease undergoing therapy. Also provided are packaged formulations and instructions for use of the tablet, capsule, soft gel capsule, elixir, etc. Typically, the dosage requirement is between about 1 to 4 dosages a day.

Methods of Making and Testing Exemplary Formulations:

The present application also provides methods (e.g., processes) of making the formulations and compositions of the present application. Such methods are well known to those of ordinary skill in the art and can be located in the US Pharmacopeia.

Preparation of Emulsions:

Coarse emulsions are first processed with a high shear homogenizer, and then subjected to ultra-sonication to further reduce droplet size.

Turbidity Measurements:

Turbidity of the emissions is measured with an Oakton T-100 Turbidity meter form OAKTON Instruments, Vernon Hills, US.

The pre-drying emulsion (or emulsion) of the present application may include about 0.1% by weight to about 99% by weight additive or carrier, wherein the additive or carrier may also include a sweetener, a flavoring agent, a coloring agent, an anti-foaming agent, a nutrient, calcium or a calcium derivative, an energy-generating additive, an herbal supplement, a concentrated plant extract, a preservative, and/or combinations thereof.

In one aspect, the additive or carrier may include a gum and maltodextrin. In another aspect, the additive may be selected from the group consisting of crystalline cellulose, α-cellulose cross-linked carboxymethyl cellulose sodium, cross-linked starch, gelatin, casein, gum tragacanth, polyvinylpyrrolidone, chitin, chitosan, dextrin, kaolin, silicon dioxide hydrate, colloidal silicon dioxide, light silica, synthetic aluminum silicate, synthetic hydrotalcite, titanium oxide, dry aluminum hydroxy gel, magnesium carbonate, calcium carbonate, precipitated calcium carbonate, bentonite, aluminum magnesium metasilicate, calcium lactate, calcium stearate, calcium hydrogen phosphate, phosphoric acid anhydride, calcium hydrogen and talc. In one aspect, the additive comprises flowing agents selected from silicon dioxide and titanium oxide that promotes flowability or powdery characteristics of the dry powder. In one aspect, the emulsion comprises one or more additives selected from the group consisting of crystalline cellulose, α-cellulose, cross-linked carboxymethyl cellulose sodium, cross-linked starch, gelatin, casein, gum tragacanth, chitin, chitosan, calcium hydrogen phosphate, calcium hydrogen and precipitated calcium carbonate, and combinations thereof. In another aspect, the additive is comprised of wetting agents to assist in the dissolution of the dry powder, when the dry powder is dissolved in water. Such agents may include lecithin and the like.

In another aspect, the additives may include polymers that are added in an amount such that, where desired, the solution resulting from the re-dissolved powder of the present application remains substantially clear. The additive may include cellulosic polymers. Exemplary cellulosic polymers that may be used include hydroxypropyl methyl cellulose acetate, hydroxypropyl methyl cellulose, hydroxypropyl cellulose, methyl cellulose, hydroxyethyl methyl cellulose, hydroxyethyl cellulose acetate and hydroxyethyl ethyl cellulose. In another aspect, the polymers may include hydroxypropyl methyl cellulose and hydroxypropyl cellulose acetate. In another aspect, the polymers contain at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, alkoxyphthalic acid such as ethoxyphthalic acid and ethoxyisophthalic acid, and alkoxynicotinic acid such as ethoxynicotinic acid, etc.

In another aspect, exemplary cellulosic polymers may include hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, ethylbenzoic acid cellulose acetate and hydroxypropyl ethylbenzoic acid cellulose acetate. In another aspect, the cellulosic polymers may contain a non-aromatic carboxylate group, such as hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose.

In one embodiment, the composition further comprises an additive such as a sugar or sugar derivative, such as sucrose, glucose, lactose, levulose, fructose, maltose, ribose, dextrose, isomalt, sorbitol, mannitol, xylitol, lactitol, maltitol, pentatol, arabinose, pentose, xylose and galactose, and combinations thereof. The compositions of the present application may comprise from about 0.01 to 10% by weight, 10% to 25% by weight, or about 25% to 50% by weight of the above additive, relative to the weight of the dried powder formulation.

In one embodiment, the additives including coloring pigments, perfumes, flavoring and spices may be used in the appropriate concentration to obtain the desired color, flavors, aroma, taste and ultimate clarity of solution.

Drying of Stabilized Surfactants and the Cannabinoid Mixture(s):

One aspect of the drying method for the stabilized emulsion includes a spray drying method. The spray-drying method may include, for example, a method for spraying from a high-pressure nozzle. In another aspect, the method for spray-drying uses a centrifugal force, such as an atomizer. The gas or air that may be used for the spray drying includes heated air or hot air at a temperature sufficient to dry the powder having the desired moisture content. In one aspect, the gas is an inert gas such as nitrogen or nitrogen-enriched air.

In one aspect, the hot gas temperature may be at about 50 degree ° C. to 300 degree ° C., from 60 degree ° C. to 100 degree ° C., from 60 degree ° C. to 250 degree ° C., from 75 degree ° C. to 185 degree ° C., from 100 degree ° C. to 180 degree ° C., from 180 degree ° C. to 190 degree ° C., or about 180 degree ° C. The high pressure that may be used for the spray during process used in a high pressure nozzle may include about 10 to 1,000 psi, 100 to 800 psi or 200 to 500 psi. The spray drying may be carried out under conditions such that the residual water or residual moisture content of the dry powder may be controlled to about 1% to about 6%, 1% to 5%, 2% to 6%, 3% to 6% or about 3% to 5%.

In one aspect, the emulsions may then be sprayed dried in conventional spray drying equipment from commercial suppliers, such as Buchi, Niro, Yamato Chemical Co., Okawara Kakoki Co., and similar commercially available spray drier. Spray drying processes, such as rotary atomization, pressure atomization and two-fluid atomization may also be used. Examples of the devices used in these processes include Parubisu Mini-Spray GA-32 and Parubisu Spray Drier DL-41 (Yamato Chemical Co.) or Spray Drier CL-8, Spray Drier L-8, Spray Drier FL-12, Spray Drier FL-16 or Spray Drier FL-20, (Okawara Kakoki Co.), may be used for the spray drying method using rotary-disk atomizer. The nozzle of the atomizer that produces the powder of the present application may include, for example, nozzle types 1A, 1, 2A, 2, 3 (Yamato Chemical Co.) or similar commercially available nozzles, may be used for the above-mentioned spray drier. In addition, disks type MC-50, MC-65 or MC-85 (Okawara Kakoki Co.) may be used as rotary disks of the spray-drier atomizer.

In one aspect, the spray or flash drying devices traditionally used for the industrial manufacture of a milk or coffee powder may also be employed in the present method. See Jensen J. D., Food Technology, June 60-71, 1975. In one aspect, the spray drying devices may include those described in U.S. Pat. No. 4,702,799. In one embodiment, operation of the spray drier may be performed at about 200-400 degree ° C. at the end of the spray nozzle where the rest of the device may be operated at a lower temperature which may reach the air outlet temperature, such as the sprayer described in U.S. Pat. No. 3,065,076. In another aspect, the spray-drying apparatus used in the process of the present application may be any of the various commercially available apparati. Representative examples of spray drying apparati are the Anhydro Dryers (Anhydro Corp., Attleboro Falls, Mass.), the Niro Dryer (Niro Atomizer Ltd., Copenhagen, Denmark) or a Leaflash apparatus (CCM Sulzer). In one aspect, a spray-drier with a pressure nozzle may be used.

In another aspect, the powder obtained from the drying process may comprise 10% by weight, 20% by weight, 30% by weight, 40% by weight, 50% by weight, 60% by weight, 70% by weight, 80% by weight, or 90% by weight or more of particles having an average particle size in the range from about 5 to 1,000 microns, from about 10 to 500 microns, from 10 to 350 microns, from 20 to 250 microns, from 40 to 200 microns, or about 50 to 150 microns. In one aspect, the powder obtained from the drying process comprises of about 20% to 80% by weight of particles with an average particle size of 50 to 150 microns.

The dry composition of the present application may be formulated to provide a dry powder that is stable, and may form a partially turbid solution, a milky or cloudy solution, or a clear solution as desired. Where a substantially clear solution or composition is not desired, such as a milky or cloudy solution or composition is desired as obtained from the dry powder, the ratio of the solubilizing agent, such as TPGS or TPGS-750-M, to the cannabinoid mixture(s) may be reduced. For example, the ratio (w/w or wt/wt) of the emulsion, such as TPGS or TPGS-750-M, to cannabinoid mixture(s) may be reduced to a range of about 2:1 to about 1.5:1, 1.3:1, 1:1, or about 0.9:1 or less.

The dry powder formulation of the present application provides cannabinoid mixture(s) that are stable to decomposition. Without being bound by any theory presented herein, it is believed that the judicious selection of the solid support allows the encapsulation of the cannabinoid mixture(s), provides substantially no surface oil and shields the cannabinoid mixture(s) from oxidation by exposure to ambient air. In addition, the dry powder formulation is readily re-dissolved in water and forms a clear solution.

The concentrated powder may be prepared as dry preparations, such as, for example, a powder, a granular material, a crystalline material, other types of dry particle preparations or combinations thereof. In one aspect, the dry preparations may be prepared by mixing the ingredients and compositions, as disclosed herein, to form a concentrated solution, and then drying the solution to a dry powder form by conventional drying methods. Representative drying methods may include for example, lyophilization (or freeze drying), spray drying, fluid bed drying, drum drying, pulse combustion drying and various combinations thereof. In one aspect, the method is a spray drying method.

Surfactants or Solubilizing Agents:

One or more surfactants (or solubilizing agents), or a mixture of surfactants may be used in the present formulations. Representative surfactants employed may include: HLB.gtoreq.10 surfactants such as Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); HLB 8-12 such as Polysorbate 85, polyglyceryl-6-dioleate (Caprol MPGO), Mixtures of high and low HLB emulsifiers; and LB.ltoreq.8 such as Sorbitan monooleate (Span 80), Capmul MCM, Maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), propylene glycol monolaurate (e.g., Capmul PG-12 or Lauroglycol 90), polyglyceryl-3 dioleate (Plurol Oleique CC497), polyglyceryl-3 diisostearate (Plurol Diisostearique) and lecithin with and without bile salts. NAV-WeedMD CBD oil 49-38:

A mixture of THC/CBD oil (7.70 g, THC/CBD oil from NAV-WeedMD extract contains 49.2% CBD and 38.4% THC and some dark-colored impurities) is added to a 250 mL high pressure Corning glass bottle with screw-on cap and the THC/CBD oil in the glass bottle was warmed with a hot water bath (heated via a hot plate) to 60-70 degree. C. for 3 minutes.

Cremophor RH40 (50 g) was added to a separate 100 mL beaker, and the Cremophor RH40 was heated in a microwave (1000 Watts of power) at high power for about 90 seconds until the Cremophor RH40 was free flowing. 33.1 g (32.1 mL, density 1.03 g/mL at 60 degree. C.) of the heated Cremophor RH40 was added to the 250 mL bottle containing the THC/CBD oil. 4.70 g of canola oil, 1.5 g of mixed tocopherol, 0.2 g of ascorbic acid, 1.7 g of Vitamin E TPGS (tocoferosolan), 5.0 g of glycerol, and 56.2 g of distilled water was added to the 250 mL Corning glass bottle. The screw cap was screwed onto the Corning glass bottle and tightened. The glass bottle was placed into a microwave oven and heated for 3 consecutive heating and depressurization periods at high power as follows: Heating at 55 seconds followed by opening of the cap to release the pressure; heating again for 30 seconds followed by opening of the cap to release the pressure, and heating again for 20 seconds followed by opening the cap to release the pressure build-up, and then re-tighten up the cap to seal the bottle.

The bottle is then immediately placed in under running cold water (about room temperature) with moderate swirling for at least 30 seconds, and the cooling was performed at no more than about 45 seconds. After the cooling, the solution became clear and transparent, with a dark amber in color. Due to the dark color, it is not obvious whether the solution is clear and homogeneous, so the solution is diluted for visualization, as follows. 10 mL of the dark solution is then diluted with 90 mL of water to form a 100 mL diluted solution. The diluted solution shows that the THC/CBD solution is clear and homogeneous.

In cases where the solution appears opaque, cloudy or inhomogeneous, the 3-steps microwave heating at 55 seconds, 30 seconds and 20 seconds is repeated, followed by the same water cooling step is performed again to provide a clear, homogenous solution.

To the homogeneous solution is added ascorbic acid (0.3 g), sodium benzoate (0.060 g), and potassium sorbate (0.12 g), where the ascorbic acid, sodium benzoate and potassium sorbate dissolve in the homogeneous solution. The homogeneous solution is filtered through a SCILOGEX 3 μm Spare Hydrophobic Filter into a 200 mL storage container and sealed with a screw cap. The storage container is stored away from direct sunlight at room temperature.

As described herein, the methods may be used for batch processing to prepare the composition. However, continuous processing of the described methods may also be employed, using 2 feeds of liquids, a water feed containing the water soluble components of the composition, and an oil feed containing the fat soluble components of the composition. The 2 feeds are dosed in their desired proportions using suitably designed pumps (volumetric or positive displacement pumps) into small mixing tank, or into a static mixer. The combined feeds are then passed over 2 suitably designed sequentially mounted heat exchangers [HEX](i.e. plate HEX, shell and tube HEX), the first one heating the combined mixed feed to the desired engineered target temperature, the second one cooling the hot mixed feed down to below 50 degree ° C. for collection.

Emulsions prepared according to the above procedure may be dried using various drying methods as provided herein. In one embodiment, the emulsions may be dried using the spray drying methods as described herein. The spray dried composition comprises water content from about 1% to about 10%, 1% to 6%, 2% to 5%, 3% to 4%, 1% to 3%, 2% to 3%, 3% to 6%, 3% to 5%, or about 3% to 4%. Accordingly, the clarity or homogeneity of the aqueous solution containing the compositions as described herein may be controlled by the amount residual water remaining in the dried powders.

The compositions and methods of the present application are illustrated by the examples described herein. These examples are offered to illustrate, but not to limit the claimed present application.

Solubility, clarity and stability results of the solution prepared according to the procedures as described herein demonstrate that the formulations as described herein maintains clarity and stability for the desired period of time under the storage conditions. Bioavailability of Emulsions:

To determine the extent of the relative bioavailability increase of a representative composition described herein (experimental product) over a standard commercial oily solution (control product), using the same balanced source of a decarboxylated natural extract concentrate of CBD, a single center, randomized, double blind, placebo-controlled, parallel bioavailability study was conducted with 32 subjects enrolled (16 per arm). Dosage was standardized to 10 mg of THC. Primary outcomes were Area under the curve (overall absorption), maximum concentration (bioavailability), and time to maximum concentration (speed of absorption) for CBD, DELTA 9-THC, 11-OH-THC, 11-NOR-9-Carboxy-DELTA 9-THC (11-NOR-9-Carboxy-THC) and CBDA. Secondary outcome was a subjective evaluation of drug effects assessed using modified Drug Effects Questionnaire (DEQ-5) for the control product and the experimental product. Relevant experimental descriptions of several study aspects are detailed below.

Administration: A pre-dose plasma (via IV catheter) and a urine sample is collected from the participant before the administration of the emulsions/formulations. A selected emulsion formulation and comparator products are prepared as follows: (372 mg calculated according to Example 22) of the experimental product and control product, in an amount to correspond to a 10 mg target delivery of THC, is each mixed with 7 gm of plain oats (28 gm package divided into four portions to give 7 gm) prepared in water. The bowl in which the product is mixed is rinsed with 100 ml water and the participant consumes the complete amount. After the product mixture is consumed, the participant is provided with an 8 oz. glass of water that is then consumed in its entirety within 1-2 minutes.

Therapeutic methods using the emulsion and formulations of the present application may be used effectively to treat, reduce or mitigate a variety of diseases selected from the group consisting of neurological disorders and neurological diseases such as neuropathic pain, chronic pain, migraine, Post Traumatic Stress Disorder (PTSD), pruritus, rheumatoid arthritis, sleep apnea, Huntington disease (HD), Alzheimer's disease, Parkinson's disease, amyotrophic lateral sclerosis (ALS), hypertension, incontinence, diabetes, hepatitis C, multiple sclerosis (MS), rheumatoid arthritis (RA), osteoporosis, dystonia, epilepsy, fibromyalgia and Tourette syndrome (TS), gastrointestinal disorders including functional bowel diseases such as irritable bowel syndrome (IBS) and inflammatory bowel diseases such as Crohn's disease (CD) and colitis, hepatitis C and HIV infections. In another variation, there is provided a method for the treatment of nausea and vomiting associated with chemotherapy and appetite stimulation of AIDS patients suffering from the wasting syndrome, the method comprises the administration of the stabilized, aqueous and purified cannabis oil emulsion as recited herein.

Accordingly, there is provided a stabilized, aqueous and purified cannabis oil emulsion comprising: a) CBD and b) at least one emulsifier selected from the group consisting of Poloxamer 188, Polysorbate 80, Polysorbate 20, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; wherein the emulsion is stable for a period of at least 30 days when stored at about 20-30 degree. C.

In yet another aspect, the cannabis oil emulsion further comprises one or more additives comprising: a) a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof; and b) a reducing agent selected from the group consisting of L-ascorbic acid-6-palmitate, vitamin C and ubiquinol, or mixtures thereof. In another aspect, the cannabis oil emulsion further comprises a metal chelator selected from the group consisting of ethylenediaminetetraacetic acid (EDTA), disodium EDTA and calcium disodium EDTA and mixtures thereof. In another aspect, the cannabis oil emulsion further comprises an absorption enhancer or bioavailability enhancer selected from the group consisting of medium chain fatty acids, omega-3 fatty acids, capric acid, caprylic acid, (8-[2-hydroxybenzoyl]-amino)caprylic acid, N-(10-[2-hydroxybenzoyl]-amino)decanoic acid, N-(8-[2-hydroxybenzoyl]-amino)caprylic acid (SNAC, salcaprozate sodium), 8-(N-2-hydroxy-5-chloro-benzoyl)-amino-caprylic acid (5-CNAC), N-(10-[2-hydroxybenzoyl]-amino)decanoic acid, alkylglycosides, chitosan, trimethylated chitosan, protease inhibitors, β-glycoprotein inhibitors, dodecyl-2-N,N-dimethylamino propionate (DDAIP), calcium chelating agents (i.e. ethylene glycol tetraacetic acid, ethylene diamine tetraacetic acid (EDTA), salicylic acid, flavonoids (i.e. quercetin ((2-(3,4-dihydroxyphenyl)-3,5,7-trihydroxy-4Hchromen-4-one), luteolin), isoflavones (i.e. genistein (5,7-dihydroxy-3-(4-hydroxyphenyl)chromen-4-one)), flavonoid glycosides (naringin), alkaloids (i.e. sinomenine (7,8-didehydro-4-hydroxy-3,7-dimethoxy-17-methylmorphinan-6-one), triterpenoid saponins (glycyrrhizin [(3,18)-30-hydroxy-11,30-dioxoolean-12-en-3-yl 2-O-glucopyranuronosyl-Dglucopyranosiduronic acid]), nitrile glycosides, phytomolecules (i.e. lysergol, allicin (garlic)), terpenes (ginkgolide A, B, C and J), tetranortriterpinoids such as limonoids, caryophyllene, geraniol, humulene, limonene, linalool, myrcene, ocimene, pinene, terpineol, terpinolene, valanencene, ginsenosides, epigallocatechin, epigallocatechin gallate, phenanthrene, cuminumcyminum Linn, herb, ginger, aloe vera, capsaicin, colchicine, vincristine, matrine, ammonium glycyrrhizinate, beeswax, piperine, trikatu, and their pharmaceutically acceptable salts (i.e. sodium), or derivatives (i.e. esters). In another aspect of the cannabis oil emulsion, the range of the ratio of the emulsifier to cannabinoid mixture(s) is between 11.0:1.0 to 1.0:1.0, 7.0:1.0 to 1.5:1.0 or 5.0:1.0 to 2.0:1.0.

In another aspect of the cannabis oil emulsion, the cannabinoid mixture(s) concentration in the emulsion is about 10%, 9%, 8%, 7%, 5%, 3%, 2%, 1%, 0.5%, 0.1% or 0.01% or less. In another aspect of the cannabis oil emulsion, the emulsion comprises of particle size is less than about 500 nm, less than 300 nm, less than 200 nm, less than 100 nm, less than 80 nm, less than 60 nm; less than 40 nm; or between about 20 and 30 nm, as measured by DLS. In another aspect of the cannabis oil emulsion, the emulsion has a measured Nephelometric Turbidities in a range of about 10 to 1000, 20 to 300 or 30 to 100.

In another embodiment, there is provided a method for the preparation of the cannabinoid mixture(s) emulsion of the above, wherein the method comprises: a) weighing the components of the above emulsion into a reaction container; b) heating the combined emulsion to a temperature from about 25 degree ° C. to about 130 degree ° C. with agitation for a sufficient amount of time to prepare the emulsion; and c) cooling the emulsion to about 25 degree ° C. In one aspect of the above method, the preparation is performed under nitrogen atmosphere. In another aspect of the method, the heating of the cannabinoid mixture(s) emulsion is performed to a temperature of about 70 degree ° C. to 100 degree ° C., or about 80 degree ° C. to 95 degree ° C. In another aspect of the above method, the cooling of the cannabinoid mixture(s) oil emulsion is performed using an external ice bath or the equivalent. In another embodiment of the cannabinoid mixture(s) oil emulsion prepared by the above method, the resulting stable emulsion has a shelf stability of at least 3 months, 6 months or 12 months when stored at about 0 degree ° C. to 50 degree ° C., or about 25 degree ° C. to 35 degree ° C. In another aspect of the cannabis oil emulsion, the natural odor of the cannabinoid mixture(s) emulsion is effectively masked and provides a pleasant taste for oral consumption. In another aspect of the cannabinoid mixture(s) emulsion, the oxidative stability of the emulsion is enhanced over that of a cannabinoid mixture(s) by at least 3 months when the cannabinoid mixture(s) is stored at about 0 degree ° C. to 50 degree ° C.

In another embodiment, there is provided a liquid nutritional composition selected from the group consisting of beverages, soft drinks, carbonated beverages, enhanced waters, gels, gelatins, concentrates, beverage enhancers, wherein the composition is prepared by the method comprising: a) obtaining the cannabis oil emulsion as described above; and b) diluting the cannabinoid mixture(s) emulsion to a desired liquid nutritional composition.

In another embodiment, there is provided a method for treating or alleviating a neurological disorders and neurological diseases selected from neuropathic pain, chronic pain, migraine, Post Traumatic Stress Disorder (PTSD), Huntington disease (HD), Alzheimer's disease, Parkinson's disease, amyotrophic lateral sclerosis (ALS), multiple sclerosis (MS), epilepsy and dystonia to a subject in need of such treatment, the method comprising the administration to the subject in need thereof a therapeutically effective amount of the stabilized, aqueous and purified cannabis oil emulsion of any of the above recited embodiments and aspects. In another embodiment, there is provided a method for treating or alleviating a disease or disorder selected from the group consisting of pruritus, rheumatoid arthritis, sleep apnea, hypertension, incontinence, diabetes, hepatitis C, rheumatoid arthritis (RA), osteoporosis, fibromyalgia and Tourette syndrome (TS), gastrointestinal disorders including functional bowel diseases such as irritable bowel syndrome (IBS) and inflammatory bowel diseases such as Crohn's disease (CD) and colitis, hepatitis C and HIV infections to a subject in need of such treatment, the method comprising the administration to the subject in need thereof a therapeutically effective amount of the stabilized, aqueous and purified cannabis oil emulsion of any of the above recited embodiments and aspects. In yet another embodiment, there is provided a method for treating or alleviating a disease or disorder selected from the group consisting of nausea and vomiting associated with chemotherapy and appetite stimulation of AIDS patients suffering from the wasting syndrome, the method comprises the administration to the patient in need thereof the stabilized, aqueous and purified cannabinoid mixture(s) emulsion of any one of the above embodiments and aspects.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

1. A stabilized, aqueous and purified cannabis oil emulsion comprising: a) CBD and b) at least one emulsifier selected from the group consisting of Tween 80, Tween 60, Tween 20, Span 80, Span60, Span 20, Poloxamer 188, Polysorbate 80, Polysorbate 20, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; wherein the emulsion is stable for a period of at least 30 days when stored at about 20-30 degree. C.

2. A method of treating one or more neurological disorders comprising:
administering a therapeutically effective amount of a stabilized, aqueous and purified cannabis oil emulsion.

3. The method of claim 2 wherein the one or more neurological disorders is selected from the group consisting of: neuropathic pain, chronic pain, migraine, Post Traumatic Stress Disorder (PTSD), Huntington disease (HD), Alzheimer's disease, Parkinson's disease, amyotrophic lateral sclerosis (ALS), multiple sclerosis (MS), epilepsy and dystonia.

4. The method of claim 2, wherein the cannabis oil emulsion further comprises a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof.

5. The method of claim 2, wherein the cannabis oil emulsion further comprises a reducing agent selected from the group consisting of L-ascorbic acid-6-palmitate, vitamin C and ubiquinol, or mixtures thereof.

6. The method of claim 2, wherein the cannabis oil emulsion further comprises a metal chelator selected from the group consisting of ethylenediaminetetraacetic acid (EDTA), disodium EDTA and calcium disodium EDTA and mixtures thereof.

7. The method of claim 2, wherein the cannabis oil emulsion further comprises an absorption enhancer.

8. A method of preparing a cannabinoid mixture emulsion, wherein the method comprises:
a) weighing the components of the above emulsion into a reaction container;
b) heating the cannabinoid mixture emulsion to a temperature from about 25 degree ° C. to about 130 degree ° C. with agitation for a sufficient amount of time to prepare the emulsion; and
c) cooling the emulsion to about 25 degree ° C.

9. The method of claim 8, wherein the method is performed under nitrogen atmosphere.

10. The method of claim 8, wherein the heating of the cannabinoid mixture emulsion is performed to a temperature of about 70 degree ° C. to 100 degree ° C.

11. The method of claim 8, wherein the cooling of the cannabinoid mixture emulsion is performed using an external ice bath.

12. The method of claim 8, wherein the resulting cannabinoid mixture emulsion has a shelf stability of at least 12 months when stored at about 0 degree ° C. to 50 degree ° C.
